(19) United States
(12) Patent Application Publication
(10) Pub. No.: US 2018/0140286 A1
(43) Pub. Date: May 24, 2018

(54) MRI GUIDED BREAST BIOPSY TARGETING ASSEMBLY WITH OBTURATOR OVERSHOOT FEATURE

(71) Applicant: Devicor Medical Products, Inc., Cincinnati, OH (US)

(72) Inventors: Robert M. Householder, Loveland, OH (US); Andrew P. Nock, Dayton, OH (US); Edward A. Rhad, Fairfield, OH (US)

(21) Appl. No.: 15/571,636

(22) PCT Filed: May 3, 2016
(86) PCT No.: PCT/US16/30517

§ 371 Date: Nov. 3, 2017

Related U.S. Application Data
(60) Provisional application No. 62/157,586, filed on May 6, 2015.

(51) Int. Cl. A61B 10/02; A61B 17/34

(52) U.S. Cl. CPC A61B 10/0233; A61B 2090/034; A61B 17/3423

(57) ABSTRACT

An apparatus for use with a biopsy device to position a needle of the biopsy device within breast tissue is described and claimed. The apparatus includes a cannula, an obturator; and an obturator actuation assembly, which is described as an “overshoot obturator”. The overshoot obturator is designed to give the physician the ability to set the z-lock to the targeted depth, then move the obturator forward, relative to the sleeve to cut additional tissue. This reduces the tissue force which can push the obturator out of the desired biopsy location.

### FIELD OF THE INVENTION

The present invention relates generally to vacuum-assisted breast biopsy devices for use in breast biopsy procedures using MRI.

### BACKGROUND

Biopsy samples have been obtained in a variety of ways in various medical procedures including open and percutaneous methods using a variety of devices. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device. Biopsy devices may be used under stereotactic guidance, ultrasound guidance, MRI guidance, Positron Emission Mammography (“PEM” guidance), Breast-Specific Gamma Imaging (“BSGI”) guidance or otherwise.

The state of the art technology for conducting a breast biopsy is to use a vacuum-assisted breast biopsy device. A current textbook in this area is “Vacuum-Assisted Breast Biopsy with Mammotome®”, available Nov. 11, 2012, copyright 2013 by Devicor Medical Germany Gumby, published in Germany by Springer Medicine Verilog, Authors: Markus Hahn, Anne Tardyon and Jan Cassel man, ISBN 978-3-642-34270-7, http://www.amazon.com/Vacuum-Assisted-Breast-Biopsy-Mammotome-Diagnostic/dp/3642342701?ie=UTF8&keywords=vacuum %20assisted%20breast%20biopsy%20with%20Mammotome&qid=1460663723&ref=sr_1_1&sr=8-1.

Biopsy samples have been obtained in a variety of ways in various medical procedures using a variety of devices. Biopsy devices may be used under stereotactic guidance, ultrasound guidance, MRI guidance, PEM guidance, BSGI guidance, or otherwise. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device.

Known biopsy devices and biopsy system components are disclosed in U.S. Pat. No. 5,526,822, entitled “Method and Apparatus for Automated Biopsy and Collection of Soft Tissue,” issued Jun. 18, 1996; U.S. Pat. No. 5,928,164, entitled “Apparatus for Automated Biopsy and Collection of Soft Tissue,” issued Jul. 27, 1999; U.S. Pat. No. 6,017,316, entitled “Vacuum Control System and Method for Automated Biopsy Device,” issued Jan. 25, 2000; U.S. Pat. No. 6,086,544, entitled “Control Apparatus for an Automated Surgical Biopsy Device,” issued Jul. 11, 2000; U.S. Pat. No. 6,162,187, entitled “Fluid Collection Apparatus for a Surgical Device,” issued Dec. 19, 2000; U.S. Pat. No. 6,432,065, entitled “Method for Using a Surgical Biopsy System with Remote Control for Selecting an Operational Mode,” issued Aug. 13, 2002; U.S. Pat. No. 6,626,849, entitled “MRI Compatible Surgical Biopsy Device,” issued Sep. 11, 2003; U.S. Pat. No. 6,752,768, entitled “Surgical Biopsy System with Remote Control for Selecting an Operational Mode,” issued Jun. 22, 2004; U.S. Pat. No. 7,442,171, entitled “Remote Thumbwheel for a Surgical Biopsy Device,” issued Oct. 8, 2008; U.S. Pat. No. 7,648,466, entitled “Manually Rotatable Piercer,” issued Jan. 19, 2010; U.S. Pat. No. 7,837,632, entitled “Biopsy Device Tissue Port Adjustment,” issued Nov. 23, 2010; U.S. Pat. No. 7,854,706, entitled “Clutch and Valving System for Tetherless Biopsy Device,” issued Dec. 1, 2010; U.S. Pat. No. 7,914,464, entitled “Surgical Biopsy System with Remote Control for Selecting an Operational Mode,” issued Mar. 29, 2011; U.S. Pat. No. 7,938,786, entitled “Vacuum Timing Algorithm for Biopsy Device,” issued May 10, 2011; U.S. Pat. No. 8,083,687, entitled “Tissue Biopsy Device with Rotatable Linked Thumbwheel and Tissue Sample Holder,” issued Dec. 21, 2011; U.S. Pat. No. 8,118,755, entitled “Biopsy Sample Storage,” issued Feb. 1, 2012; U.S. Pat. No. 8,206,316, entitled “Tether less Biopsy Device with Reusable Portion,” issued on Jun. 26, 2012; U.S. Pat. No. 8,241,226, entitled “Biopsy Device with Rotatable Tissue Sample Holder,” issued on Aug. 14, 2012; U.S. Pat. No. 8,251,916, entitled “Revolving Tissue Sample Holder for Biopsy Device,” issued Aug. 28, 2012; U.S. Pat. No. 8,454,531, entitled “Icon-Based User Interface on Biopsy System Control Module,” published May 21, 2009, issued on Jun. 4, 2013; U.S. Pat. No. 8,532,747, entitled “Biopsy Marker Delivery Device,” issued Sep. 10, 2013; U.S. Pat. No. 8,702,623, entitled “Biopsy Device with Discrete Tissue Chambers,” issued on Apr. 22, 2014; U.S. Pat. No. 8,764,680, entitled “Handheld Biopsy Device with Needle Firing,” issued on Jun. 11, 2014; U.S. Pat. No. 8,801,742, entitled “Needle Assembly and Blade Assembly for Biopsy Device,” issued Aug. 12, 2014; U.S. Pat. No. 8,858,465, entitled “Biopsy Device with Motorized Needle Firing,” issued Oct. 14, 2014; U.S. Pat. No. 8,938,285, entitled “Access Chamber and Markers for Biopsy Device,” issued Jan. 20, 2015; U.S. Pat. No. 9,095,326, entitled “Biopsy System with Vacuum Control Module,” issued Aug. 4, 2015 and U.S. Pat. No. 9,095,326, entitled “Biopsy System with Vacuum Control Module,” issued Aug. 4, 2015. The disclosure of each of the above-cited U.S. patents is incorporated by reference herein.

Additional known biopsy devices and biopsy system components are disclosed in U.S. Pat. Pub. No. 2006/0074345, entitled “Biopsy Apparatus and Method,” published Apr. 6, 2006 and now abandoned; U.S. Pat. Pub. No. 2008/0214955, entitled “Presentation of Biopsy Sample by Biopsy Device,” published Sep. 4, 2008; U.S. Pat. Pub. No. 2009/0131821, entitled “Graphical User Interface for Biopsy System Control Module,” published May 21, 2009, now abandoned; U.S. Pat. Pub. No. 2010/0152610, entitled “Hand Actuated Tether less Biopsy Device with Pistol Grip,” published Jun. 17, 2010, now abandoned; U.S. Pat. Pub. No. 2010/0160819, entitled “Biopsy Device with Central Thumbwheel,” published Jun. 24, 2010, now abandoned; U.S. Pat. Pub. No. 2013/0053724, entitled “Biopsy Device Tissue Sample Holder with Bulk Chamber and Pathology Chamber,” published Feb. 28, 2013, will issue on May 3, 2016 as U.S. Pat. No. 9,326,755; U.S. Pat. Pub. No. 2013/0144188, entitled “Biopsy Device with Slide-In Probe,” published Jun. 6, 2013; and U.S. Pat. Pub. No. 2013/0324882, entitled “Control for Biopsy Device,” published Dec. 5, 2013. The disclosure of each of the above-cited U.S. patent application Publications, U.S. Non-Provisional patent applications, and U.S. Provisional patent applications is incorporated by reference herein.

A known localization mechanism used for guiding a core biopsy instrument is disclosed in U.S. Pat. No. 7,507,210, entitled “Biopsy Cannula Adjustable Depth Stop,” issued Mar. 24, 2009, the disclosure of which is incorporated by reference herein. The localization mechanism includes a grid plate configured to removable receive a guide cube capable of supporting and orienting an MRI-compatible biopsy instrument. For instance, a combination of an obturator and targeting cannula/sleeve may be introduced through a breast to a biopsy site via the guide cube, with proper positioning confirmed using MRI imaging. The obturator may then be removed and the needle of a biopsy device may then be inserted through the targeting cannula/sleeve to reach the targeted lesion.

In U.S. Pat. Pub. No. 2005/0283069, entitled “MRI Biopsy Device Localization Fixture” published Dec. 22, 2005, the disclosure of which is incorporated by reference herein, a localization mechanism, or fixture, is described that is used in conjunction with a breast coil for breast compression and for guiding a core biopsy instrument during prone biopsy procedures in both open and closed Magnetic Resonance Imaging (MRI) machines. The localization fixture includes a three-dimensional Cartesian position able guide for supporting and orienting an MRI-compatible biopsy instrument, and, in particular, a cannula/sleeve to a biopsy site of suspicious tissues or lesions. Another merely illustrative localization mechanism used for guiding a core biopsy instrument is disclosed in U.S. Pat. No. 7,507,210, entitled “Biopsy Cannula Adjustable Depth Stop,” issued Mar. 24, 2009, the disclosure of which is incorporated by reference herein. The localization mechanism includes a grid plate configured to removably receive a guide cube capable of supporting and orienting an MRI-compatible biopsy instrument. For instance, a combination of an obturator and targeting cannula/sleeve may be introduced through a breast to a biopsy site via the guide cube, with proper positioning confirmed using MRI imaging. The obturator may then be removed and the needle of a biopsy device may then be inserted through the targeting cannula/sleeve to reach the targeted lesion.

In U.S. Pat. No. 7,831,290, issued Oct. 20, 2010, the disclosure of which is incorporated by reference herein, a localization mechanism, or fixture, is described that is used in conjunction with a breast coil for breast compression and for guiding a core biopsy instrument during prone biopsy procedures in both open and closed Magnetic Resonance Imaging (MRI) machines. The localization fixture includes a three-dimensional Cartesian positionable guide for supporting and orienting an MRI-compatible biopsy instrument, and, in particular, a cannula/sleeve to a biopsy site of suspicious tissues or lesions.

A Z-stop may enhance accurate insertion, and prevent over-insertion or inadvertent retraction of a biopsy device targeting cannula/sleeve and obturator. In particular, a Z-stop may engage the localization fixture or cube at a distance from the patient set to restrict the depth of insertion of a biopsy device needle into a patient. Known Z-stop devices are disclosed in U.S. Pat. No. 7,507,210, entitled “Biopsy Cannula Adjustable Depth Stop,” issued Mar. 24, 2009, the disclosure of which has been previously incorporated by reference herein.

A known problem with breast biopsy is when the obturator “overshoots” the targeted tissue within the breast due to a “snowplowing” effect of having the obturator push away the breast tissue. Correcting for overshoot in the past has been achieved by unlocking the Z-stop and manually moving the obturator and sleeve forward to cut the targeted tissue. The obturator and sleeve would then be manually moved back to the biopsy site and the Z-stop would be reset. It is recognized that it is not optimal during a MRI breast biopsy procedure to have to have the Z-stop first locked, then unlocked and then relocked, during the procedure. It would be desirable to have equipment that would not make it necessary to do this.

While several systems and methods have been made and used for obtaining a biopsy sample, it is believed that no one prior to the inventor has made or used the invention described in the appended claims.

### SUMMARY OF THE INVENTION

The first aspect of the instant claimed invention is an apparatus for use with a biopsy device to position a needle of the biopsy device within breast tissue, the apparatus comprising: a cannula comprising an open distal end and a longitudinal lumen, wherein the longitudinal lumen extends through the cannula and is in communication with the open distal end, wherein the longitudinal lumen is sized to receive the needle of the biopsy device; an obturator sized for insertion into the longitudinal lumen of the cannula, the obturator having a distal end extending from the open distal end of the cannula when the obturator is inserted into the cannula, and the obturator having a recess proximate to the distal end of the obturator; and an obturator actuation assembly, wherein the obturator actuation assembly is secured to a proximal end of the obturator, wherein the hub assembly is configured to selectively actuate the obturator between a first position and a second position relative to the cannula.

The second aspect of the instant claimed invention is an apparatus for use with a biopsy device to position a needle of the biopsy device within breast tissue, the apparatus comprising: a cannula comprising an open distal end, wherein the cannula further defines a lateral opening, wherein the lateral opening is proximate to the open distal end of the cannula, wherein the cannula further comprises a longitudinal lumen communicating with the lateral opening and the open distal end, wherein the longitudinal lumen is sized to receive the needle of the biopsy device; an obturator sized for insertion into the longitudinal lumen of the cannula, the obturator having a sharp distal end extending from the open distal end of the cannula when the obturator is inserted into the cannula, and the obturator having a recess proximate to the distal end of the obturator; and a hub assembly, wherein the hub assembly is secured to the distal end of the cannula and the obturator, wherein the hub assembly is configured to selectively drive the obturator between a first position and a second position relative to the cannula.

The third aspect of the instant claimed invention is a method for positioning an obturator and cannula into tissue of a patient, the method comprising the steps of: inserting the obturator into a lumen defined by the cannula such that a sharp distal tip of the obturator protrudes through an open distal end of the cannula, wherein the act of inserting the obturator into the lumen of the cannula defines a first distance between the sharp distal tip of the obturator and the open distal end of the cannula; piercing tissue of a patient by inserting the obturator into tissue of the patient, wherein the obturator is inserted into tissue of the patient while the obturator is disposed within the lumen of the cannula; and actuating the obturator relative to the cannula to drive the obturator toward a second position, wherein the obturator in the second position defines a second distance between the sharp distal tip of the obturator and the open distal end of the cannula, wherein the second distance is greater than the first distance.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a perspective view of an exemplary alternative targeting set for use with the biopsy system of FIG. 1;

FIG. 2 depicts a partial perspective view of an obturator and obturator actuation assembly of the targeting set of FIG. 1;

FIG. 3 depicts an exploded view of the obturator and obturator actuation assembly of FIG. 2;

FIG. 4 depicts a perspective view of an external member the obturator actuation assembly of FIG. 2;

FIG. 5 depicts a perspective cross-sectional view of the external member of FIG. 4, with the cross-section taken along line 5-5 of FIG. 4;

FIG. 6 depicts a perspective view of an internal member of the obturator actuation assembly of FIG. 2;

FIG. 7 depicts a cross-sectional view of the obturator actuation assembly of FIG. 2, with the cross-section taken along line 7-7 of FIG. 2;

FIG. 8 depicts a cross-sectional view of the obturator actuation assembly of FIG. 2, with the cross-section taken along line 8-8 of FIG. 2;

FIG. 9 depicts a perspective view of the obturator and the obturator actuation assembly of FIG. 2, with the obturator actuation assembly in an unlocked position;

FIG. 10 depicts a perspective view of the obturator and the obturator actuation assembly of FIG. 2, with the obturator actuation assembly in an advanced position;

FIG. 11 depicts a side cross-sectional view of the obturator and the obturator actuation assembly of FIG. 2, with the obturator actuation assembly in an initial position;

FIG. 12 depicts a side cross-sectional view of the obturator and the obturator actuation assembly of FIG. 2, with the obturator actuation assembly in the unlocked position;

FIG. 13 depicts a side cross-sectional view of the obturator and the obturator actuation assembly of FIG. 2, with the obturator actuation assembly in the advanced position;

FIG. 14 depicts a perspective view of another exemplary alternative targeting set for use with the biopsy system of FIG. 1;

FIG. 15 depicts a perspective view of an obturator and obturator actuation assembly of the targeting set of FIG. 14;

FIG. 16 depicts an exploded view of the obturator and obturator actuation assembly of FIG. 15;

FIG. 17 depicts a perspective view of an external member the obturator actuation assembly of FIG. 15;

FIG. 18 depicts a perspective cross-sectional view of the external member of FIG. 17, with the cross-section taken along line 18-18 of FIG. 17;

FIG. 19 depicts a perspective view of an internal member of the obturator actuation assembly of FIG. 15;

FIG. 20 depicts a cross-sectional view of the obturator actuation assembly of FIG. 15, with the cross-section taken along line 20-20 of FIG. 15;

FIG. 21 depicts a cross-sectional view of the obturator actuation assembly of FIG. 15, with the cross-section taken along line 21-21 of FIG. 15;

FIG. 22 depicts a perspective view of the obturator and the obturator actuation assembly of FIG. 15, with the obturator actuation assembly in an unlocked position;

FIG. 23 depicts a perspective view of the obturator and the obturator actuation assembly of FIG. 15, with the obturator actuation assembly in an advanced position;

FIG. 24 depicts a side cross-sectional view of the obturator and the obturator actuation assembly of FIG. 15, with the obturator actuation assembly in an initial position;

FIG. 25 depicts a side cross-sectional view of the obturator and the obturator actuation assembly of FIG. 15, with the obturator actuation assembly in the unlocked position;

FIG. 26 depicts a side cross-sectional view of the obturator and the obturator actuation assembly of FIG. 15, with the obturator actuation assembly in the advanced position;

FIG. 27 depicts a perspective view of still another exemplary alternative targeting set for use with the biopsy system of FIG. 1;

FIG. 28 depicts a perspective view of an obturator and obturator actuation assembly of the targeting set of FIG. 27;

FIG. 29 depicts an exploded view of the obturator and obturator actuation assembly of FIG. 28;

FIG. 30 depicts a perspective view of an external member the obturator actuation assembly of FIG. 28;

FIG. 31 depicts a perspective view of an internal member of the obturator actuation assembly of FIG. 28;

FIG. 32 depicts a cross-sectional view of the internal member of FIG. 31, with the cross-section taken along line 32-32 of FIG. 31;

FIG. 33 depicts a cross-sectional view of the internal member of FIG. 31, with the cross-section taken along line 33-33 of FIG. 15;

FIG. 34 depicts a perspective view of the obturator and the obturator actuation assembly of FIG. 28, with the obturator actuation assembly in an unlocked position;

FIG. 35 depicts a perspective view of the obturator and the obturator actuation assembly of FIG. 28, with the obturator actuation assembly in an advanced position;

FIG. 36 depicts a front cross-sectional view of the obturator and the obturator actuation assembly of FIG. 28, with the obturator actuation assembly in an initial position;

FIG. 37 depicts a front cross-sectional view of the obturator and the obturator actuation assembly of FIG. 28, with the obturator actuation assembly in the unlocked position;

FIG. 38 depicts a side cross-sectional view of the obturator and the obturator actuation assembly of FIG. 28, with the obturator actuation assembly in the unlocked position; and

FIG. 39 depicts a side cross-sectional view of the obturator and the obturator actuation assembly of FIG. 28, with the obturator actuation assembly in the advanced position.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

An apparatus for use with a biopsy device to position a needle of the biopsy device within breast tissue is described and claimed. The apparatus includes a cannula, an obturator; and an obturator actuation assembly, which is described as an “overshoot obturator”. The overshoot obturator is designed to give the physician the ability to set the z-lock to the targeted depth, then move the obturator forward, relative to the sleeve to cut additional tissue. This reduces the tissue force which can push the obturator out of the desired biopsy location.

The overshoot obturator provides the ability for the physician to move the obturator forward relative to the sleeve to cut a pre-set distance of additional breast tissue. A spring return then moves the obturator back into the biopsy location. The purpose of the overshoot is to reduce “snow plowing” where the obturator tip displaces the targeted tissue within the breast and the targeted tissue is not located within the biopsy bowl of the obturator.

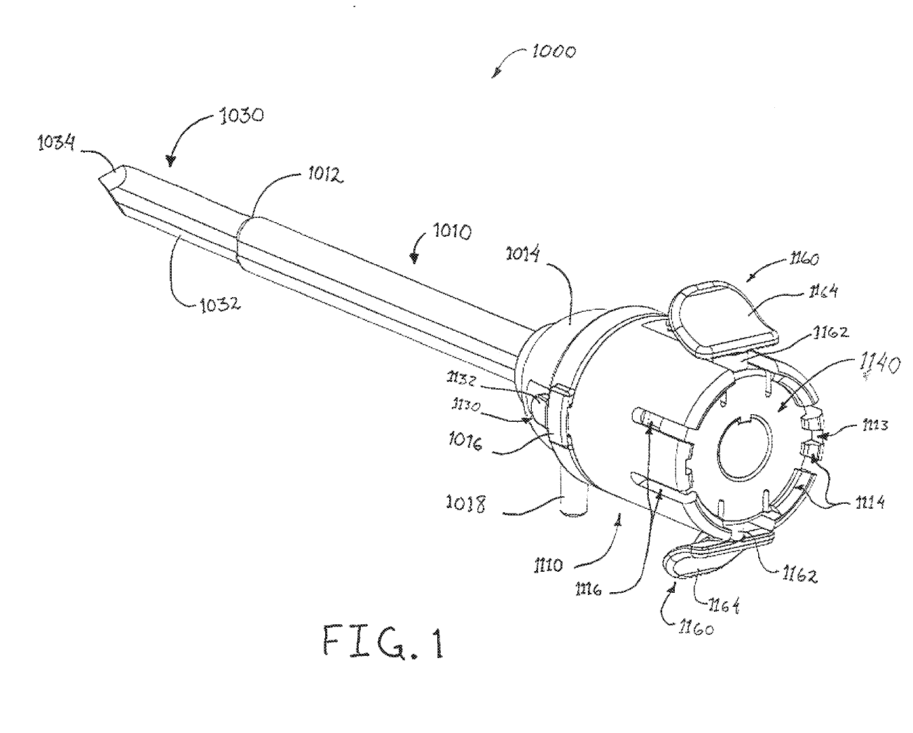

FIGS. 1, 2, 3, 4, 5, 6, 7, 8 and 9 of U.S. Pat. No. 7,507,210, incorporated by reference in its entirety, depict a perspective view of a biopsy system including a control module remotely coupled to a biopsy device, and including a localization fixture with a lateral grid plate used in conjunction with a rotatable cube to position an obturator or a probe of the biopsy device to a desired insertion depth as set by a ring stop.

During the breast biopsy procedure, typically the patient's breasts hang pendulously respectively into breast apertures on the examination table. For convenience, herein a convention is used for locating a suspicious lesion by Cartesian coordinates within breast tissue referenced to localization fixture and to thereafter selectively position an instrument, such as needle of probe that is engaged to holster portion to form biopsy device.

To enhance hands-off use of biopsy system, especially for repeated re-imaging within the narrow confines of a closed bore MRI machine, biopsy system may also guide obturator encompassed by cannula. Depth of insertion is controlled by a depth stop device longitudinally positioned on either needle or cannula. Alternatively, depth of insertion may be controlled in any other suitable fashion.

In typical MRI breast biopsy procedures, a targeting set comprising cannula and obturator is associated with a probe. In particular the obturator is slid into cannula and the combination is guided through guide cube to the biopsy site within the breast tissue. Obturator is then withdrawn from cannula, then the needle of the probe is inserted in cannula, and then biopsy device is operated to acquire one or more tissue samples from the breast via needle.

FIGS. 1-7 show an example of the targeting set (1000) of the instant claimed invention. Targeting set (1000) is for use in association with probe as similarly described above with respect to targeting set. Like with previously known targeting sets, targeting set (1000) of the present example comprises a cannula (1010), an obturator (1030), and an obturator actuation assembly (1100). As will be described in greater detail below, targeting set (1000) is generally configured such that obturator (1030) is independently actuatable relative to cannula (1010) while cannula (1010) remains longitudinally fixed via a depth stop member (not shown).

Cannula (1010) of the present example defines a lumen (not shown) and includes an open distal end (1012). The proximal end of cannula (1010) is fixedly secured to a hub (1014). Hub (1014) includes attachment features (1016) and a port (1018). As will be described in greater detail below, attachment features (1016) couple hub (1014) to a portion of obturator actuation assembly (1100). Port (1018) is in communication with the lumen defined by cannula (1010). Port (1018) may be optionally coupled to a fluid source for delivery of therapeutic substances, saline, or other fluids to a biopsy site via the lumen. It should be understood that port (1018) is merely optional and may be omitted in some examples. While not shown, it should be understood that hub (1014) includes other features and/or components such as seals, thumbwheels, fluid channels, and/or additional lumen similar to cylindrical hub described in U.S. Pat. No. 7,507,210.

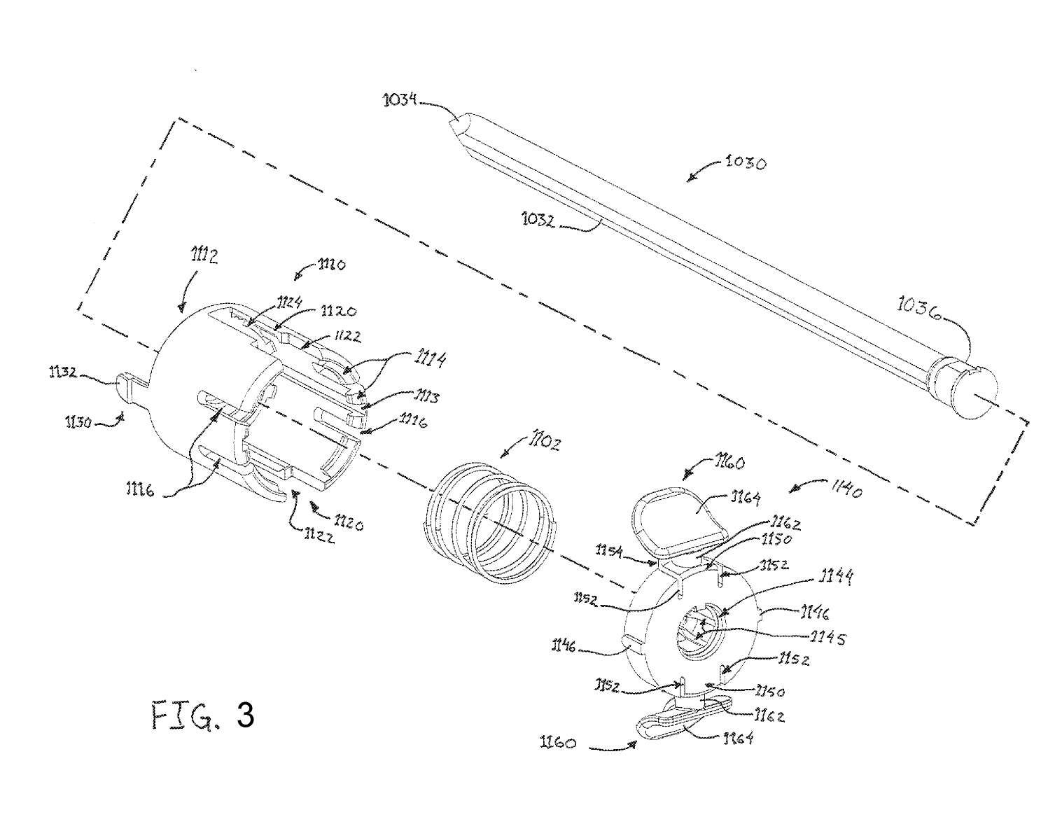

Obturator (1030) of the present example comprises a solid elongate shaft (1032) having a sharp distal tip (1034) and an ovular transverse cross-section. Shaft (1032) of the present example comprises a single MRI compatible material such as ceramic or plastic, although no such limitation is intended. For instance, in other examples shaft (1032) comprises a non-MRI compatible material such as metal. However, in such examples, obturator (1030) may be removed from cannula (1010) during an MRI imaging procedure. As can best be seen in FIG. 3, the proximal end of obturator (1030) includes an attachment feature (1036). As will be described in greater detail below, attachment feature (1036) is configured to be received by at least a portion of obturator actuation assembly (1100) such that obturator (1030) is fixedly secured relative to at least a portion of obturator actuation assembly (1100).

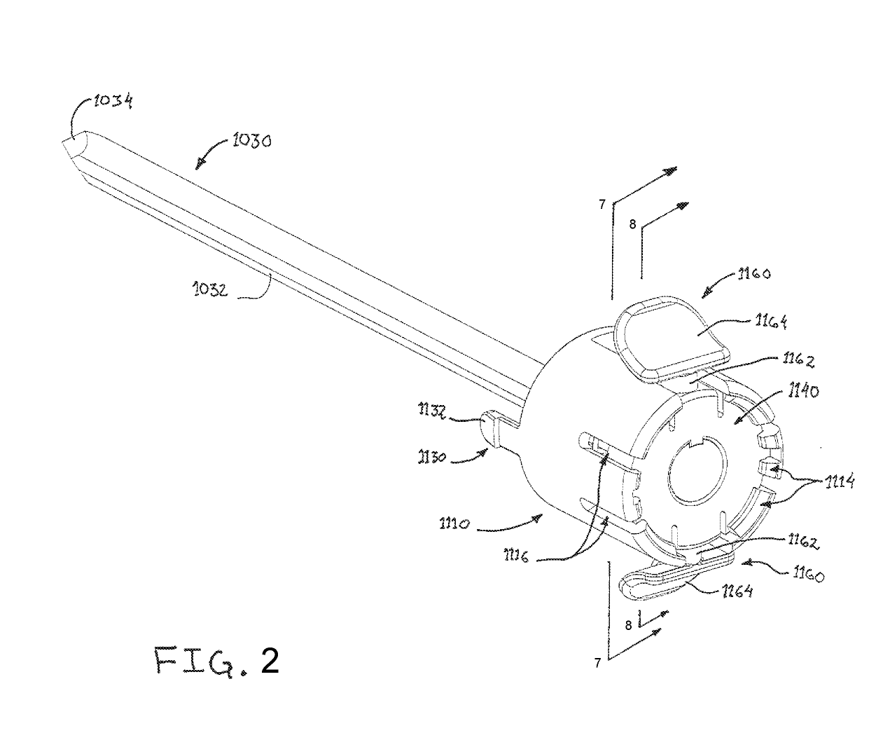

FIGS. 2-8 show obturator (1030) and obturator actuation assembly (1100). As can be seen in FIG. 2, obturator actuation assembly (1100) generally comprises an external member (1110), a spring (1102), and an internal member (1140). As will be described in greater detail below, internal member (1140) is generally selectively movable relative to external member (1110) to selectively actuate obturator (1030) relative to cannula (1010).

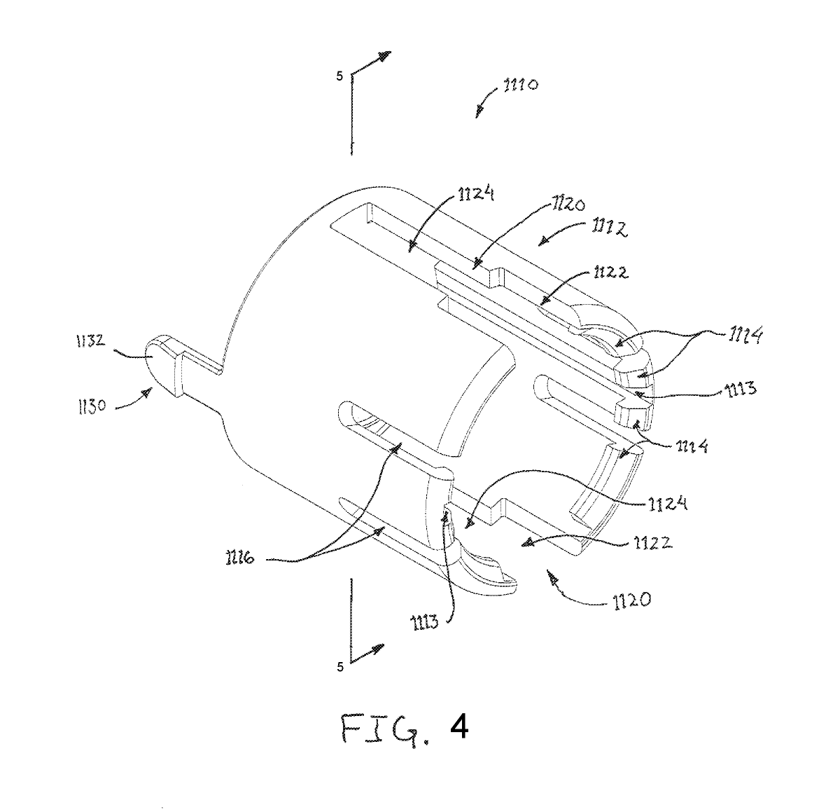

External member (1110) is shown in FIG. 4. As can be seen, external member (1110) comprises a generally cylindrical body (1112) and a pair of arms (1130) extending distally from body (1112). Body (1112) is generally configured to receive internal member (1140) such that internal member (1140) is selectively movable relative to external member (1110) as will be described in greater detail below.

The proximal end of body (1112) is open and includes a plurality of retaining features (1114) extending inwardly about the inner diameter of body (1112). As will be understood, retaining features (1114) are configured to bear against a portion of internal member (1140) to retain internal member (1140) within external member (1110). For assembly purposes, body (1112) comprises a plurality of slots (1116, 1120) extending distally from the proximal end of body (1112). Slots (1116, 1120) are configured such that retaining features (1114) are resiliently biased towards the position shown in FIG. 4. Yet retaining features (1114) are movable to permit insertion of internal member (1140) into external member (1110) during assembly.

Slots (1116, 1120) comprise four insertion slots (1116), and two actuation slots (1120). Insertion slots (1116) are configured for the functional assembly feature described above. Actuation slots (1120), in contrast, are configured for the additional purpose of facilitating actuation of internal member (1140), as will be described in greater detail below. Each actuation slot (1120) comprises a neutral portion (1122) and an actuation portion (1124). As will be understood, neutral portion (1122) is configured to contain a portion of internal member (1140) while internal member (1140) is in a proximal position. As will also be understood, actuation portion (1124) is configured to permit selective actuation of internal member (1140) to a distal position.

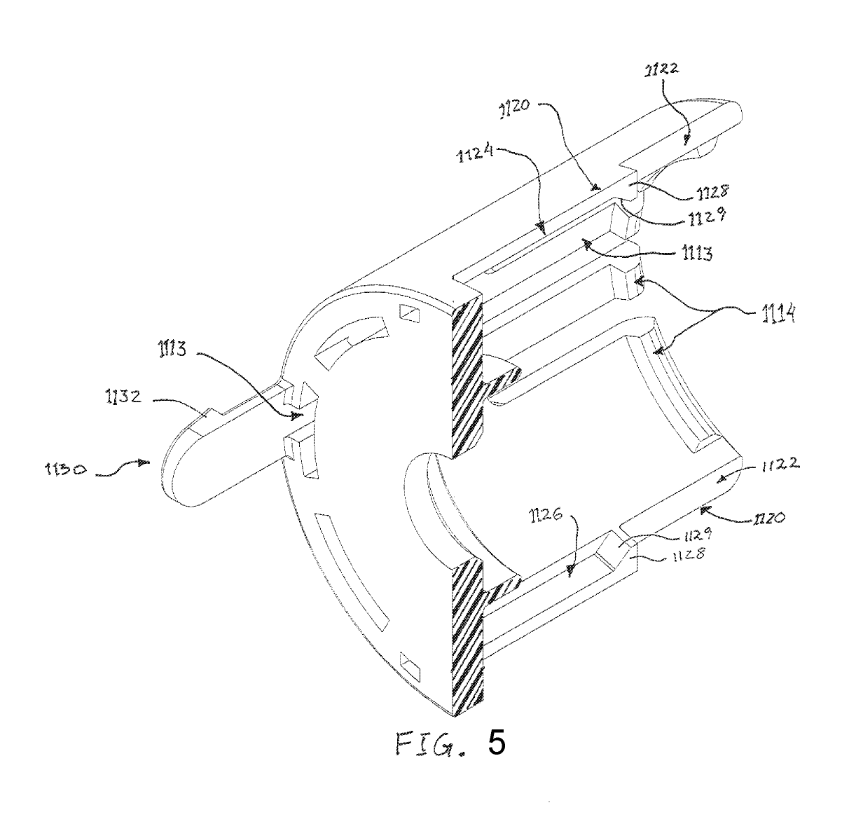

As can best be seen in FIG. 5, actuation portion (1124) defines an actuation channel (1126) on each longitudinal side of actuation portion (1124). At the proximal end of actuation portion (1124), actuation portion (1124) includes a lock member (1128) extending inwardly into actuation channel (1126). As will be described in greater detail below, lock member (1128) is configured to engage a portion of internal member (1140) to prevent inadvertent actuation of internal member (1140) or otherwise lock internal member (1140) in the proximal position. The proximal end of each lock member (1128) includes a ramped surface (1129). As will be described in greater detail below, ramped surface (1129) aids in returning internal member (1140) to the proximal position. It should be understood that while each lock member (1128) of the present example includes a ramped surface (1129), such a feature is merely optional and may be omitted in some aspects of the instant claimed invention.

Body (1112) further includes a pair of channels (1113) disposed in the inner diameter of body (1112). As can be seen in FIGS. 4 and 5, channels (1113) extend longitudinally through the length of body (1112). As will be described in greater detail below, channels (1113) are configured to receive a corresponding portion of internal member (1140) to maintain internal member (1140) in a fixed angular position relative to external member (1110).

As described above, external member (1110) includes a pair of arms (1130) extending distally from body (1112). Arms (1130) are configured to engage with attachment features (1016) disposed on hub (1014) of cannula (1010). In particular, each arm (1130) includes outwardly extending teeth (1132) that are resiliently biased to fasten arms (1130) into attachment features (1016). Thus it should be understood that arms (1130) secure external member (1110) to cannula (1010) such that movement of external member (1110) and cannula (1010) is fixed relative to each other.

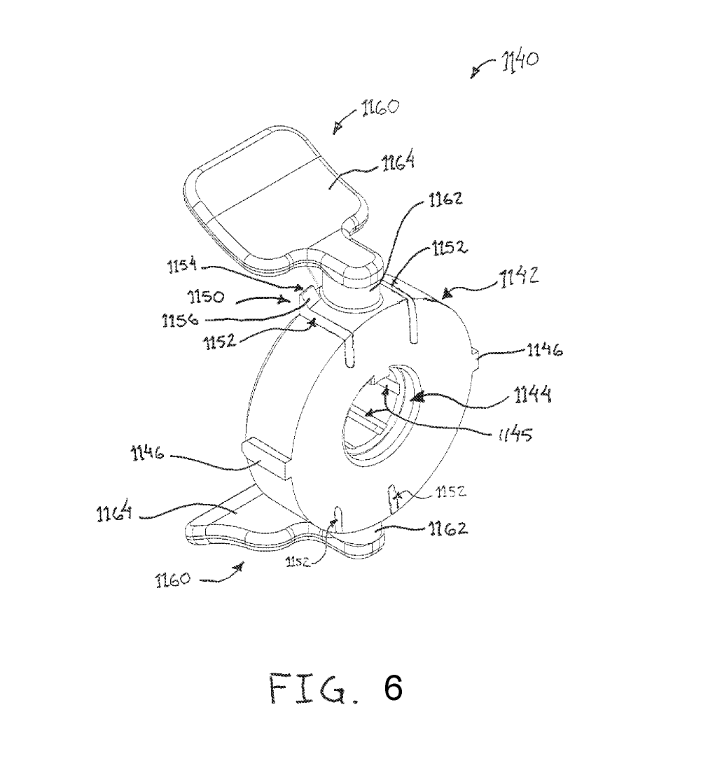

As can best be seen in FIG. 6, internal member (1140) a generally solid cylindrical body (1142) and a pair of actuation arms (1160) extending outwardly from body (1142). Body (1142) comprises a central bore (1144), a pair of indexing features (1146), and two actuation portions (1150). Central bore (1144) is configured to receive obturator (1030). In particular, central bore (1144) includes a plurality of obturator engagement features (1145) that are configured to engage with attachment feature (1036) of obturator (1030) such that obturator (1030) is fixedly secured to body (1142).

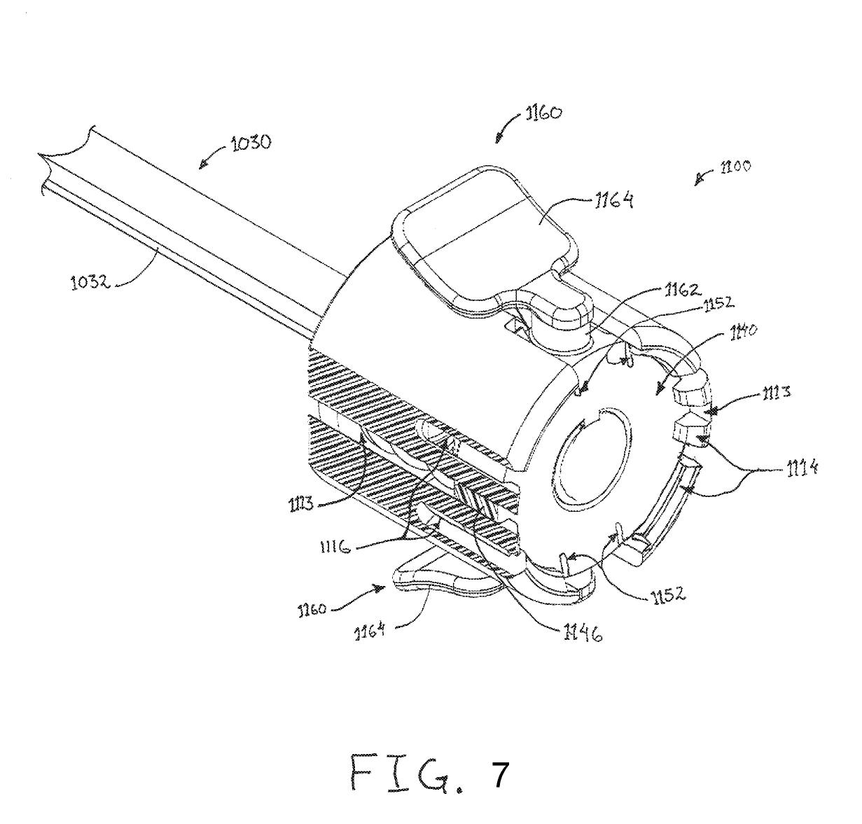

Indexing features (1146) extend outwardly from body (1142) and are configured to engage with external member (1110) to fix the angular position of internal member (1140) relative to external member (1110). As can be best seen in FIG. 7, indexing features (1146) are configured to be received within channels (1113) of external member (1110). Thus, it should be understood that indexing features (1146) are free to slide longitudinally within channels (1113) of external member (1110); yet indexing features (1146) are laterally fixed thereby preventing angular movement of internal member (1140) relative to external member (1110).

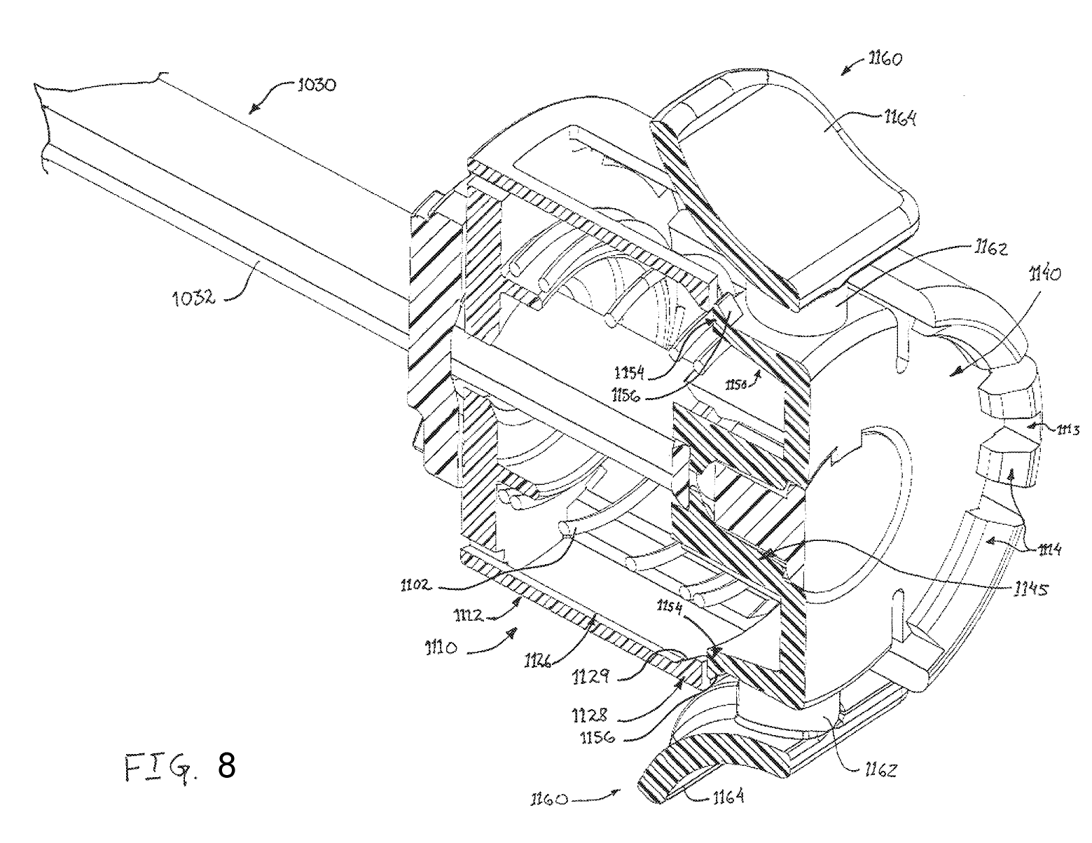

As best seen in FIGS. 6 and 8, each actuation portion (1150) is defined by a pair of slots (1152) extending through body (1142). Each actuation portion (1150) includes a lock member (1154) extending outwardly from actuation portion (1150) adjacent to each slot (1152) and the distal end of body (1142). Like with lock member (1128) described above with respect to external member (1110), each lock member (1154) of each actuation portion (1150) includes a ramped surface (1156). As will be understood, slots (1152) permit each actuation portion (1150) to flex or otherwise resiliently bend relative to body (1142) such that each lock member (1154) may disengage from each corresponding lock member (1128) of external member (1110).

Each actuation arm (1160) extends outwardly from a respective actuation portion (1150) of body (1142). As will be described in greater detail below, each actuation arm (1160) is generally configured to permit an operator to deflect actuation portion (1150) to thereby disengage each lock member (1154) of actuation portion (1150) from each corresponding lock member (1128) of external member (1110). Each actuation arm (1160) comprises a base (1162) and a grip (1164). Base (1162) is configured to extend through external member (1110) such that the respective grip (1162) is positioned externally relative to external member (1110). Grip (1164) is configured for gripping by an operator to actuate actuation arm (1160) to a desired position as will be described in greater detail below.

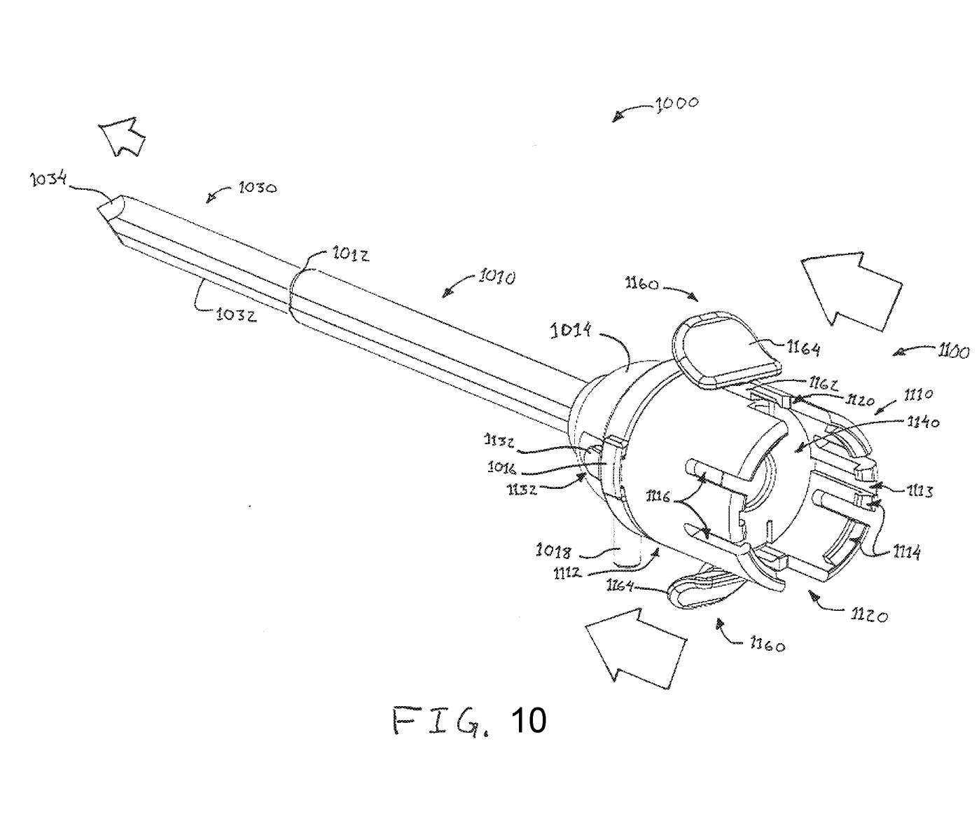

FIGS. 1 and 9-13 show an exemplary use of obturator actuation assembly (1100) to actuate obturator (1030) relative to cannula (1010). In particular, as can be seen in FIG. 1, obturator actuation assembly (1100) may begin in an initial state where obturator (1030) is in a proximal position relative to cannula (1010). In the initial state an operator may use targeting set (1000) in conjunction with depth stop device and guide cube to penetrate into tissue of a patient to a desired initial depth, as similarly described above with respect to targeting set.

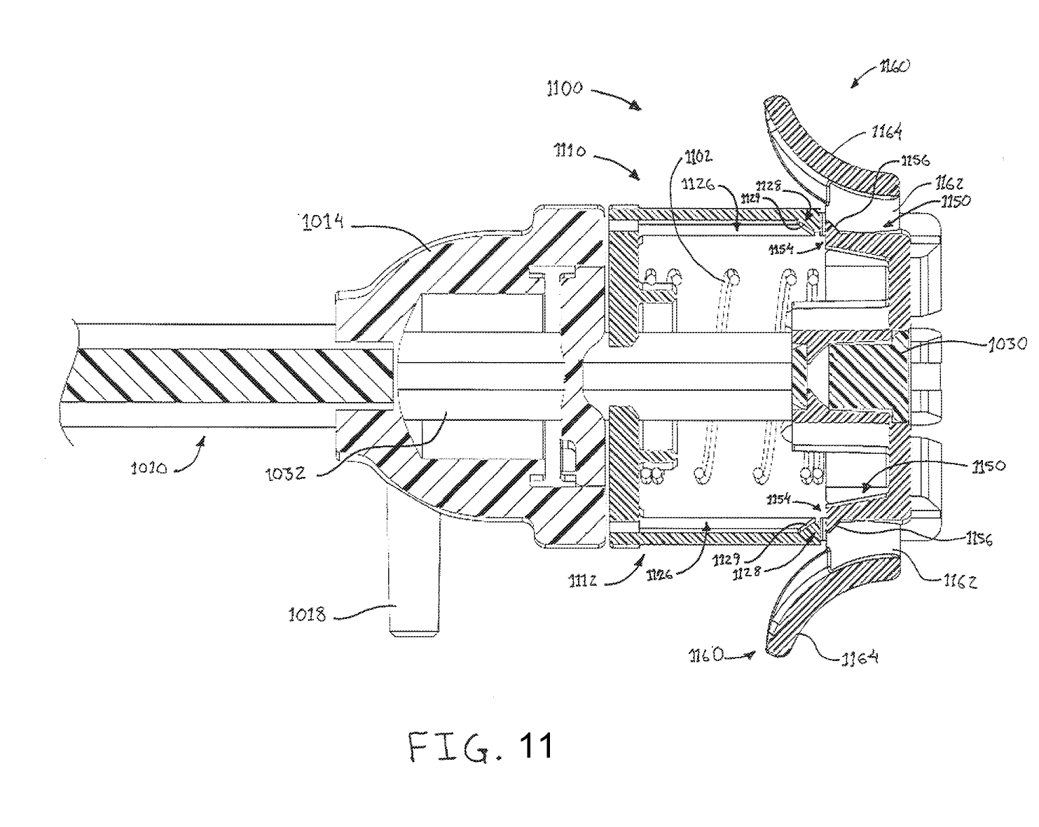

FIG. 11 shows the various components of obturator actuation assembly (1100) when in the initial position. As can be seen, internal member (1140) is initially disposed in a proximal position relative to external member (1110). With internal member (1140) disposed proximally within external member (1110), each lock member (1154) of internal member (1140) is engaged with a respective lock member (1128) of external member (1110). Thus, when internal member (1140) is in the proximal position internal member (1140) is selectively locked in position to prevent distal movement of obturator (1030) relative to cannula (1010).

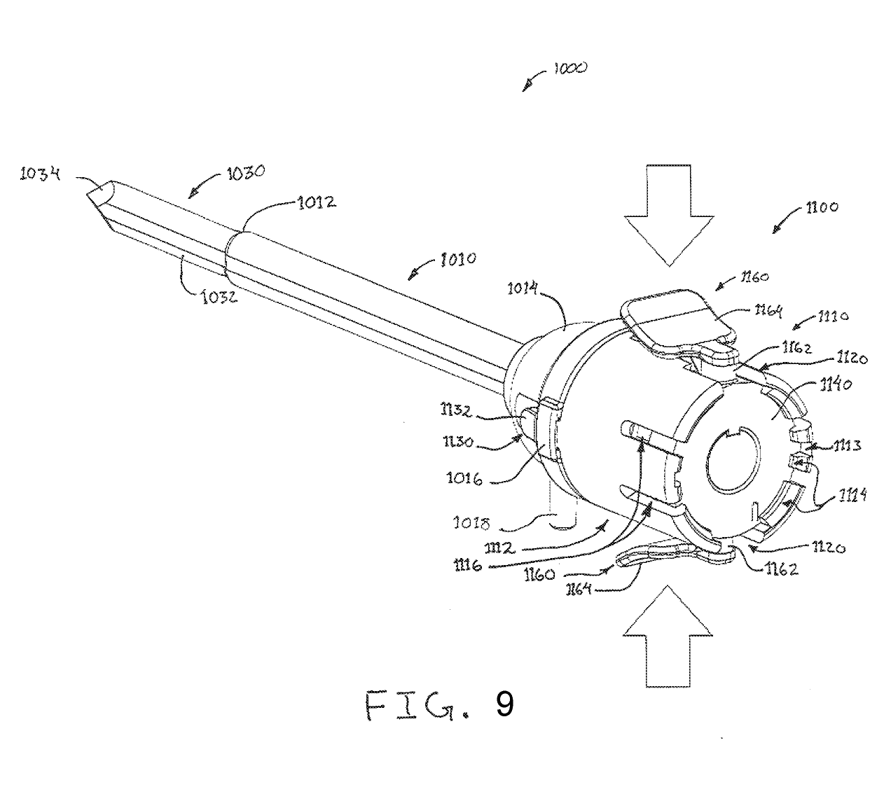

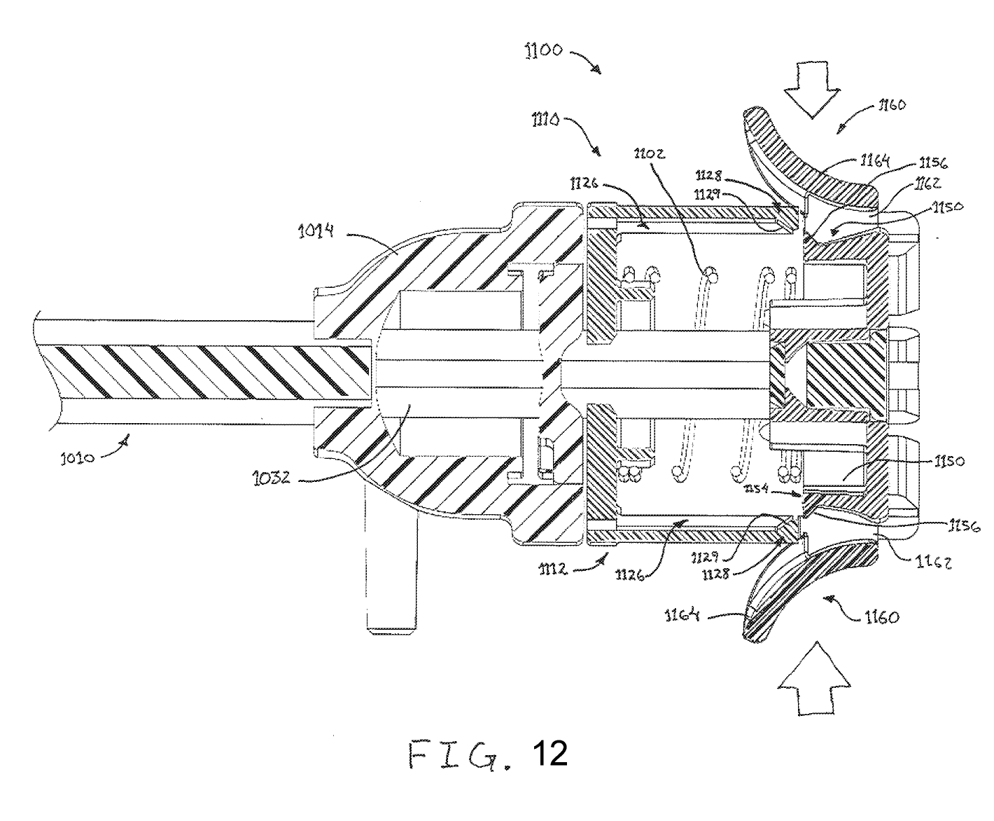

Once an operator has positioned targeting set to the desired depth within tissue of a patient, the operator may desire to further advance obturator (1030) into the tissue of the patient. To advance obturator (1030) relative to cannula (1010) using obturator actuation assembly (1100), an operator may initiate actuation by first unlocking relative movement between obturator (1030) and cannula (1010). To unlock such movement, an operator may apply a force to grips (1164) of each actuation arm (1160) as shown in FIG. 9. Such a force directs grips (1164) towards a central longitudinal axis of obturator actuation assembly (1100) thereby causing each actuation portion (1150) of internal member (1140) to correspondingly deflect. As can be seen in FIG. 12, such a deflection of each actuation portion (1150) causes each lock member (1154) of each actuation portion (1150) to move out of engagement with each respective lock member (1128) of external member (1110). With lock members (1154) of actuation portion (1150) disengaged from lock members (1128) of external member (1110), internal member (1140) is in an unlocked state and is free to move distally relative to external member (1110).

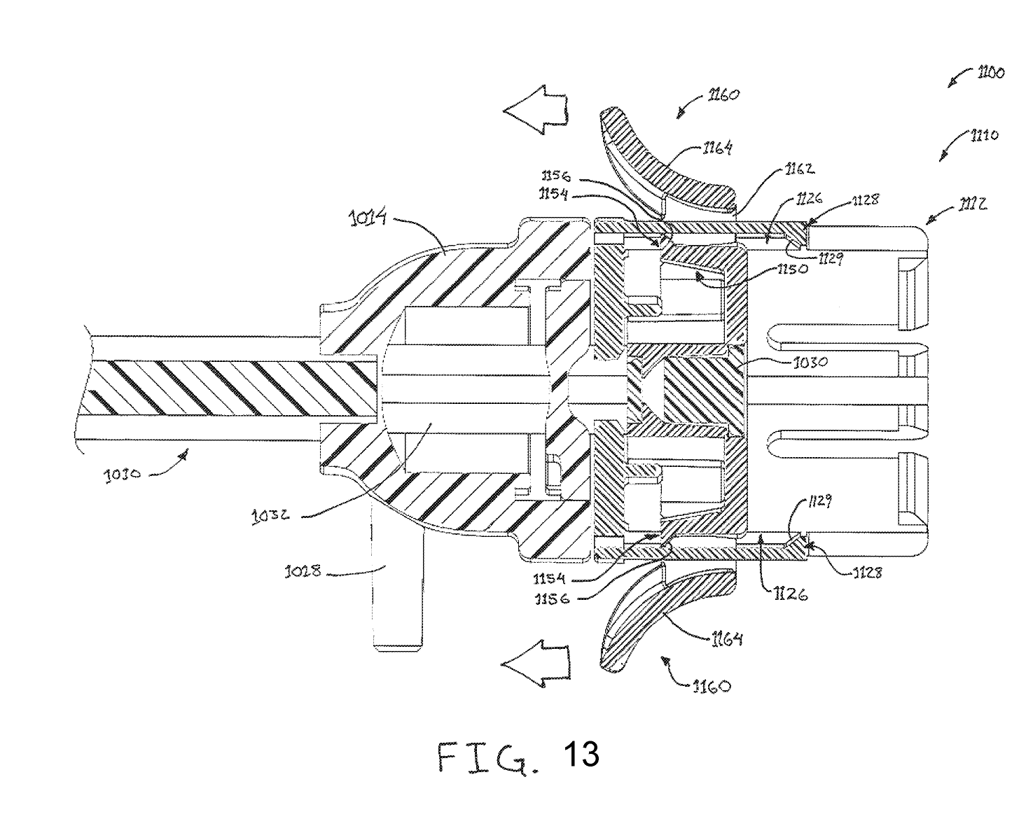

Once internal member (1140) is in an unlocked state, an operator may force internal member (1140) distally by applying a distal force to grips (1164) as shown in FIGS. 1 and 13. Such a force will cause internal member (1140) to advance obturator (1030) distally relative to cannula (1010). It should be understood that a suitable distal force will be sufficient to overcome any force supplied by spring (1102), which is disposed between internal member (1140) and external member (1110). An operator may advance internal member (1140) any suitable distance to a maximum predetermined distance defined by the length of external member (1110). Once a desired distance of actuation has been reached (or the maximum distance), an operator may optionally hold internal member (1140) in position.

Once an operator desires to return actuation assembly (1100) to the initial position, the operator may release grips (1164). Spring (1102), disposed between internal member (1140) and external member (1110), will then force internal member (1140) proximally to the position shown in FIGS. 1 and 11. It should be understood that as internal member (1140) returns to the proximal position, each respective ramped surface (1129, 1156) of each lock member (1128, 1154) engages to return internal member (1140) to a locked state. It should also be understood that while the present example uses spring (1120) to return internal member (1140) to the proximal position, no such limitation is intended. For instance, in some examples spring (1102) may be omitted and internal member (1140) may be manually actuated in both directions.

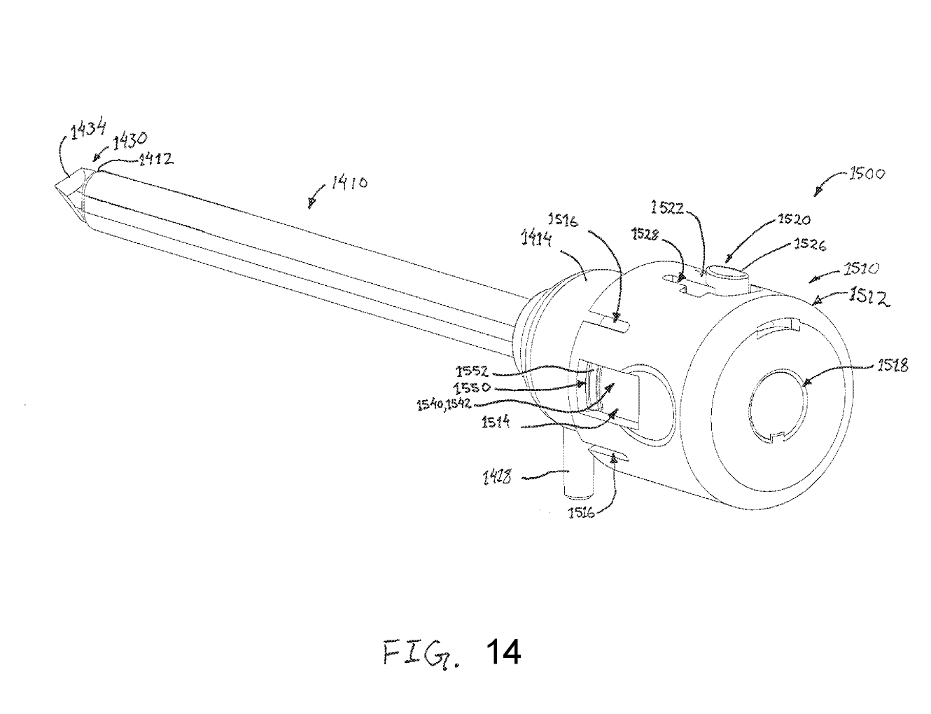

FIGS. 14-21 show another aspect of the instant claimed invention. FIGS. 14-21 show targeting set (1400) for use in association with a probe as similarly described above with respect to targeting set. Targeting set (1400) of this aspect of the instant claimed invention comprises a cannula (1410), an obturator (1430), and an obturator actuation assembly (1500). As will be described in greater detail below, targeting set (1400) is generally configured such that obturator (1430) is independently actuatable relative to cannula (1410) while cannula (1410) remains longitudinally fixed via a depth stop member (not shown).

Cannula (1410) of the present example defines a lumen (not shown) and includes an open distal end (1412). The proximal end of cannula (1410) is fixedly secured to a hub (1414). Hub (1414) includes attachment features (not shown) and a port (1418). As will be described in greater detail below, the attachment features couple hub (1414) to a portion of obturator actuation assembly (1500). Port (1418) is in communication with the lumen defined by cannula (1410). Port (1418) may be optionally coupled to a fluid source for delivery of therapeutic substances, saline, or other fluids to a biopsy site via the lumen. It should be understood that port (1418) is merely optional and may be omitted in some examples. While not shown, it should be understood that hub (1414) includes other features and/or components such as seals, thumbwheels, fluid channels, and/or additional lumen similar to cylindrical hub described above.

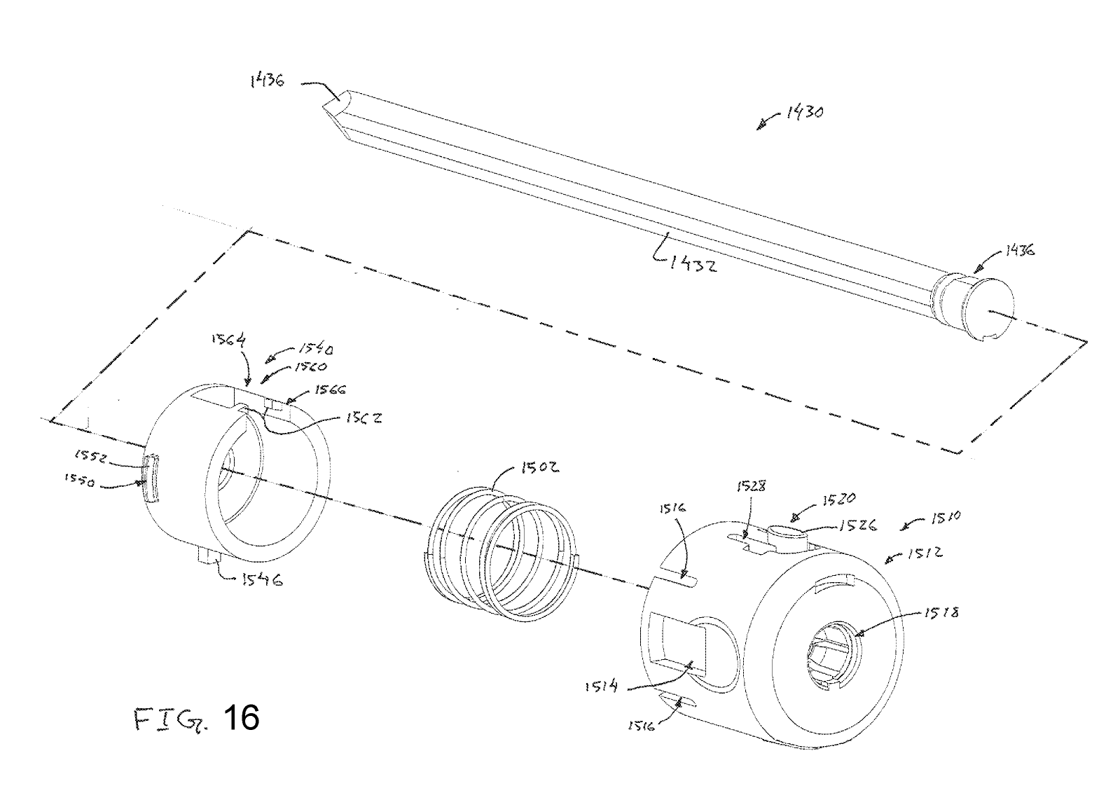

Obturator (1430) of the present example comprises a solid elongate shaft (1432) having a sharp distal tip (1434) and an ovular transverse cross-section. Shaft (1432) of the present example comprises a single MRI compatible material such as ceramic or plastic, although no such limitation is intended. For instance, in other examples shaft (1432) comprises a non-MRI compatible material such as metal. However, in such examples, obturator (1430) may be removed from cannula (1410) during an MRI imaging procedure. As can best be seen in FIG. 16, the proximal end of obturator (1430) includes an attachment feature (1436). As will be described in greater detail below, attachment feature (1436) is configured to be received by at least a portion of obturator actuation assembly (1500) such that obturator (1430) is fixedly secured relative to at least a portion of obturator actuation assembly (1500).

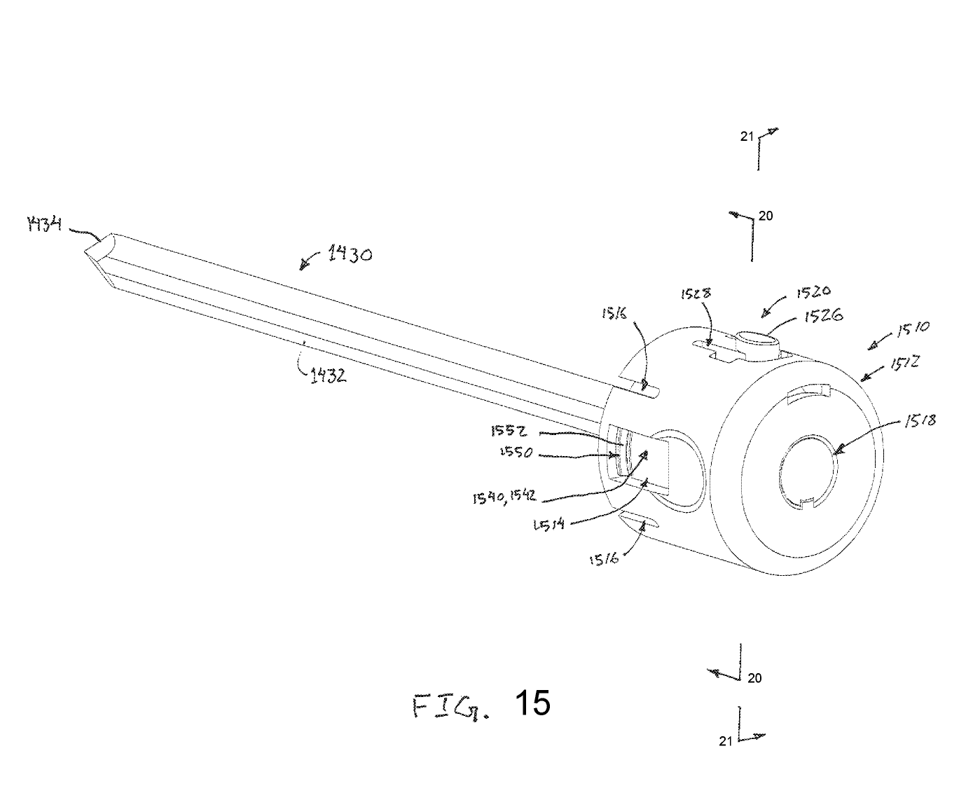

FIGS. 15-21 show obturator (1430) and obturator actuation assembly (1500). As can be seen in FIG. 15, obturator actuation assembly (1500) generally comprises an external member (1510), a spring (1502), and an internal member (1540). As will be described in greater detail below, external member (1510) is generally selectively movable relative to internal member (1540) to selectively actuate obturator (1430) relative to cannula (1410).

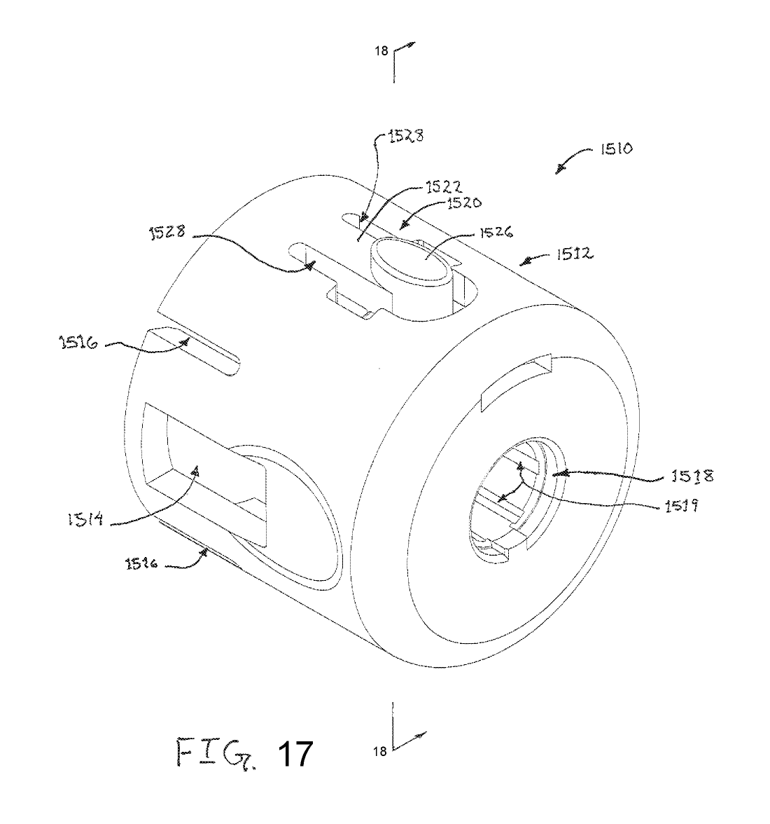

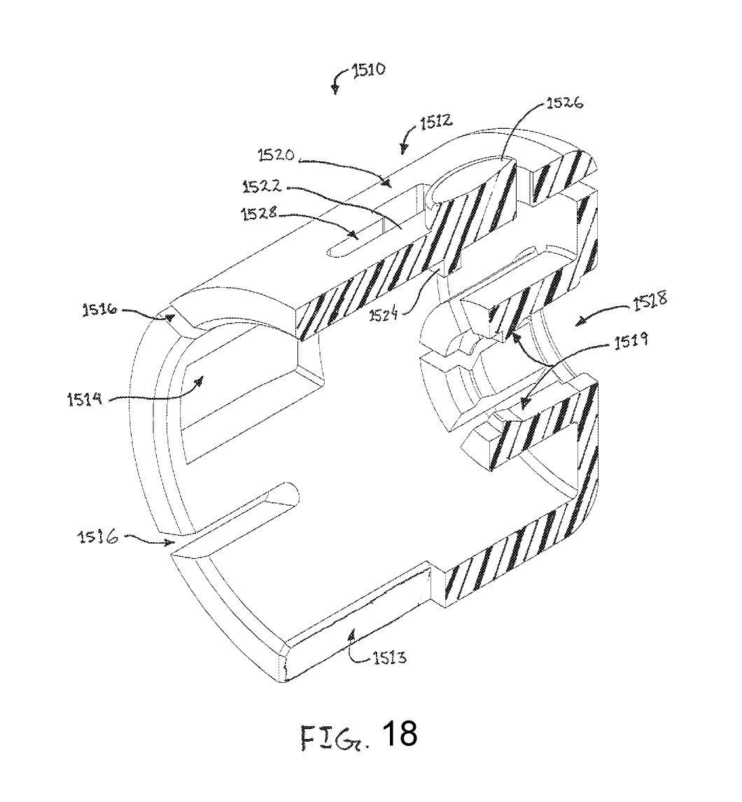

External member (1510) is shown in FIGS. 17 and 18. Unlike external member (1110) described above with respect to targeting set (1000), external member (1510) is generally configured to move relative to internal member (1540) to actuate obturator (1430). External member (1510) comprises a generally cylindrical body (1512). Body (1512) is generally configured to receive internal member (1540) such that external member (1510) is selectively movable relative to internal member (1540) as will be described in greater detail below.

The proximal end of body (1512) is substantially closed and includes a central bore (1518) disposed at the center point of body (1512). Central bore (1518) is configured to fixedly secure obturator (1430) to external member (1510). In particular, central bore (1518) includes a plurality of attachment features (1519). Attachment features (1519) are configured to engage with corresponding attachment feature (1436) of obturator (1430) to secure obturator (1430) to external member (1510).

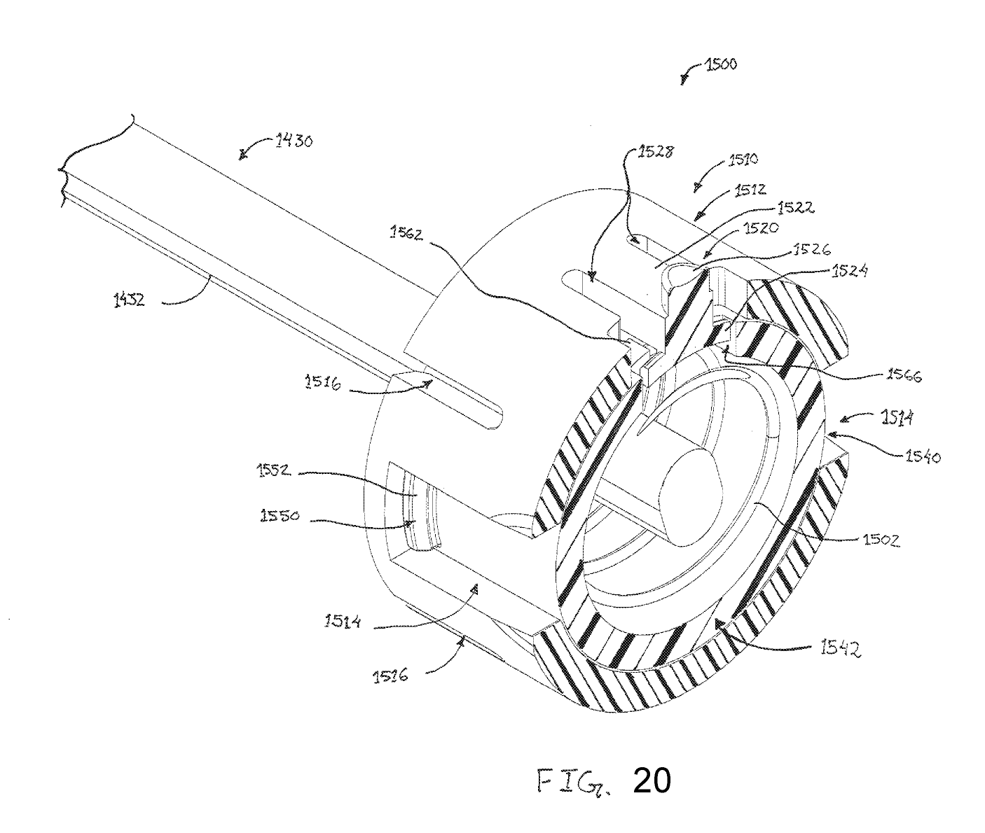

The distal end of body (1512) is open and includes a pair of retaining openings (1514) disposed on each respective side of body (1512). As will be understood, retaining openings (1514) are configured to receive a portion of internal member (1540) to retain internal member (1540) within external member (1510). For assembly purposes, body (1512) comprises a plurality of slots (1516) extending proximally from the distal end of body (1512). Slots (1516) are configured such that retaining openings (1514) are resiliently biased towards the position shown in FIG. 20. Yet the portions of body (1512) defining retaining openings (1514) are movable to permit insertion of at least a portion of internal member (1540) into retaining openings (1510) during assembly.

Body (1512) further includes a single indexing slot (1513). Indexing slot (1513) is configured to maintain external member (1510) in a fixed angular orientation relative to internal member (1540). In particular, indexing slot (1513) is configured to receive at least a portion of internal member (1540) such that external member (1513) remains slidable relative to internal member (1540), but is prevented from rotating relative to internal member (1540). It should be understood that in some examples indexing slot (1513) is omitted and slits (1516) described above are additionally operable to prevent rotation of external member (1510) relative to internal member (1540).

The upper portion of body (1512) includes an actuation lock (1520). As will be described in greater detail below, actuation lock (1520) is generally configured to selectively lock and unlock movement of external member (1510) relative to internal member (1540). Actuation lock (1520) comprises a resilient feature (1522), a lock feature (1524) and a button (1526). Resilient feature (1522) is integral with body (1512) and is defined by two intersecting actuation slots (1528) disposed in body (1512). Resilient feature (1522) is resiliently biased towards the position shown in FIG. 17. Yet, resilient feature (1522) is bendable to actuate lock feature (1524) as will be described in greater detail below. Because resilient feature (1522) is integral with body (1512) the particular resiliency or deformability of resilient feature (1522) is at least partially determined by the material of body (1512). For instance in the present example, body (1512) comprises a relatively rigid plastic exhibiting some elasticity. Accordingly, resilient member (1522) is relatively rigid, yet operable to undergo some elastic deformation. In other examples, resilient feature (1522) is a separate component from body (1512) and accordingly may have material characteristics independent of body (1512).

As can be best seen in FIG. 18, lock feature (1524) extends downwardly from resilient member (1522) and into the inner diameter of body (1512) defining a generally rectangular shape. As will be described in greater detail below, lock feature (1524) also extends from resilient member (1522) laterally to selectively engage corresponding features of internal member (1540).

Button (1526) is adjacent to lock feature (1524) and extends upwardly from resilient member (1522). Button (1526) is generally configured to be acted upon by a finger or hand of an operator such that an operator may push actuation lock (1520) downwardly. It should be understood that while button (1526) and lock feature (1524) of the present example are both integral with resilient member (1522), no such limitation is intended and in other examples button (1526) and/or lock feature (1524) may be separate components fixedly secured to resilient member (1522).

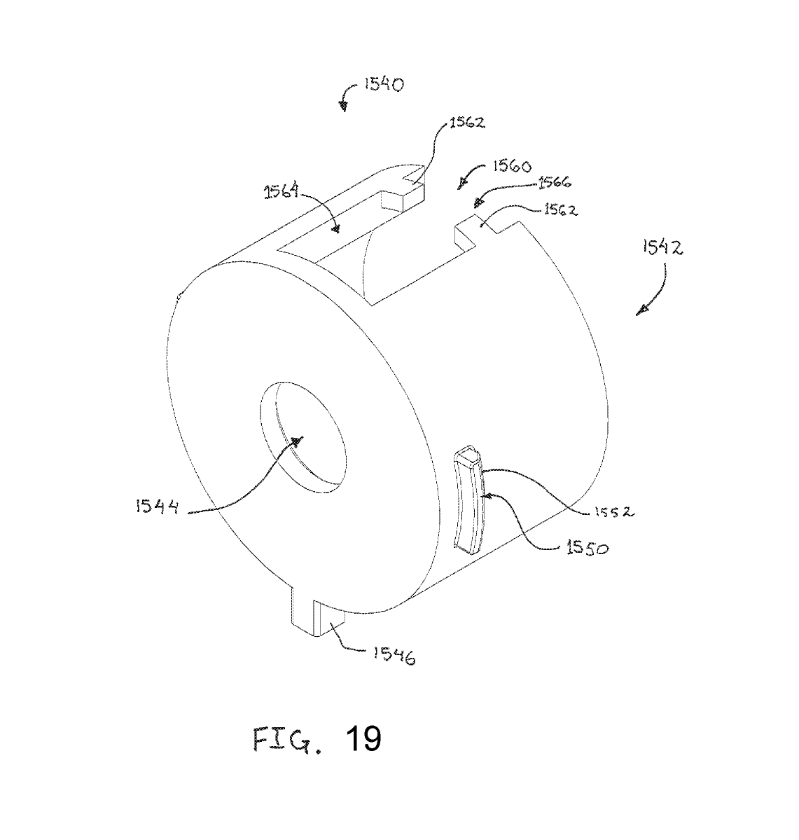

As can best be seen in FIG. 19, internal member (1540) includes a generally rigid hollow cylindrical body (1542). Body (1542) comprises a central bore (1544) disposed in the distal end of body (1542), an indexing protrusion (1546), two actuation protrusions (1550), and an actuation channel (1560). Central bore (1544) is configured to slidably receive obturator (1430). For instance, internal member (1540) of the present example is attachable to hub (1414) of cannula (1410) described above such that cannula (1410) and internal member (1540) remain fixed while obturator (1430) and external member (1510) are advanced relative to cannula (1410) and internal member (1540). Accordingly, central bore (1544) permits obturator (1430) to slidably extend through internal member (1540) for attachment to external member (1510).

Indexing protrusion (1546) extends downwardly from body (1542) and is configured to engage with indexing slot (1513) of external member (1510) to fix the angular position of internal member (1540) relative to external member (1510). Thus, it should be understood that indexing protrusion (1546) is free to slide longitudinally within indexing slot (1513) of external member (1510); yet indexing protrusion (1546) is laterally fixed thereby preventing angular movement of internal member (1540) relative to external member (1510).

Actuation protrusions (1550) similarly extend outwardly from body (1542) and are configured to engage with retaining openings (1514) of external member (1510). For ease of assembly, each actuation protrusion (1550) includes a chamfered proximal edge (1552) that is configured to engage body (1512) of external member (1510) as internal member (1540) is inserted into external member (1510). When actuation protrusions (1550) are disposed in retaining openings (1514) of external member (1510), actuation protrusions (1550) generally define the range of actuation of external member (1510) relative to internal member (1540). As will be understood, actuation protrusions (1550) are slidable within retaining openings (1514) for a certain predetermined longitudinal distance as defined by the particular length of retaining openings (1514). Additionally, actuation protrusions (1550) optionally are configured to fix the angular position of internal member (1540) relative to external member (1510) as similarly described above with respect to indexing protrusion (1546).

Actuation channel (1560), as defined by body (1542), extends distally from the proximal end of body (1512). As will be described in greater detail below, actuation channel (1560) is generally configured to engage with actuation lock (1520) of external member (1510) to permit external member (1510) to selectively move relative to internal member (1540). As can be seen, the proximal end of actuation channel (1560) is open such that actuation lock (1520) of external member (1510) may be received by actuation channel (1560). Distally of the open proximal end, actuation channel (1560) includes to lock members (1562) of integral construction with body (1542) protruding laterally into actuation channel (1560). Accordingly, lock members (1562) divide actuation channel (1560) into an actuation portion (1564) and a lock portion (1566). Actuation portion (1564) is generally of a length that corresponds to the length of movement between internal member (1540) and external member (1510). Lock portion (1566) is generally of a length suitable to receive lock feature (1524) of articulation lock (1520).

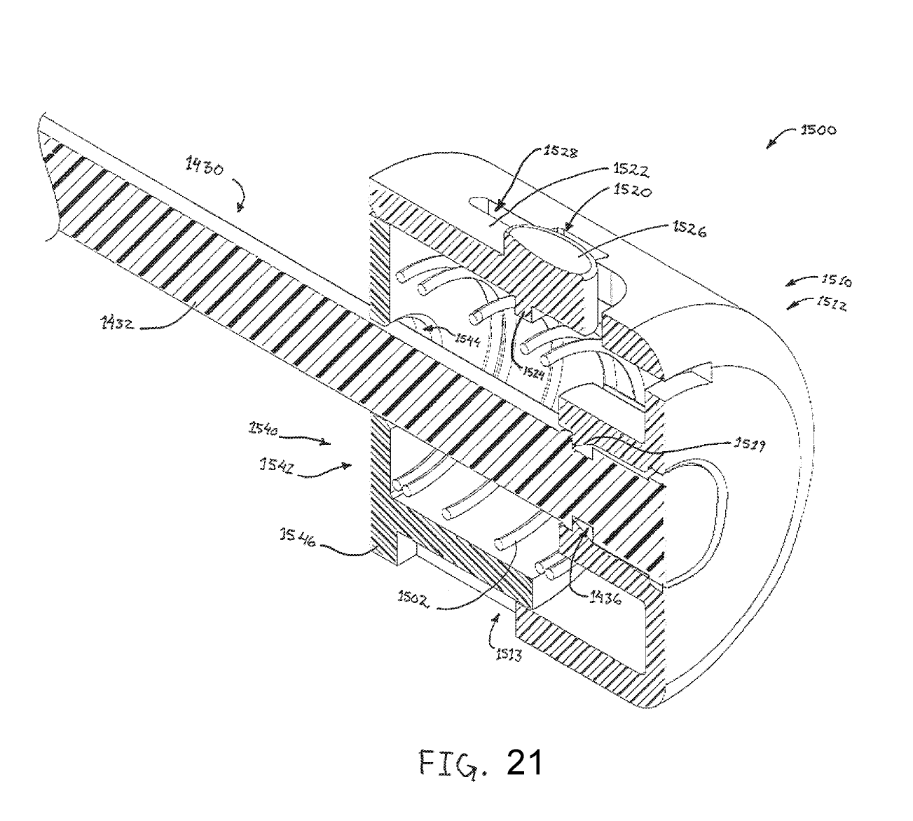

As will be described in greater detail below, lock members (1562) are configured to engage with lock feature (1524) actuation lock (1520) to selectively prevent movement of external member (1510) relative to internal member (1540). As is best seen in FIGS. 20 and 21, actuation channel (1560) is of a sufficient width to receive lock feature (1524) of actuation lock (1520). However, lock members (1562) protrude laterally into actuation channel (1560) such that at least a portion of actuation channel (1560) is narrow enough to block actuation feature (1524) of actuation lock (1520). As will be described in greater detail below, articulation lock (1520) is generally movable, bendable, and or deflectable to selectively position lock features (1524) away from lock members (1562) permitting movement of external member (1510) relative to internal member (1540).

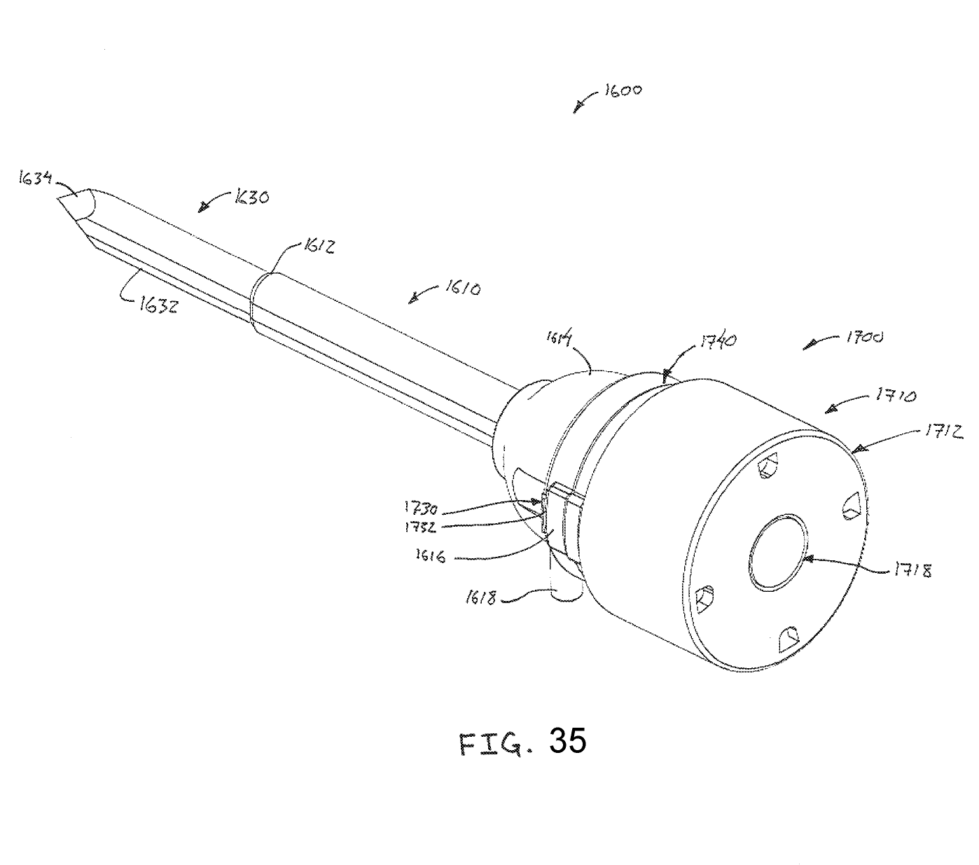

FIGS. 14 and 22-36 show an exemplary use of obturator actuation assembly (1500) to actuate obturator (1430) relative to cannula (1410). In particular, as can be seen in FIG. 14, obturator actuation assembly (1500) may begin in an initial state where obturator (1430) is in a proximal position relative to cannula (1410). In the initial state an operator may use targeting set (1400) in conjunction with a depth stop device and guide cube to penetrate into tissue of a patient to a desired initial depth, as similarly described above with respect to targeting set.

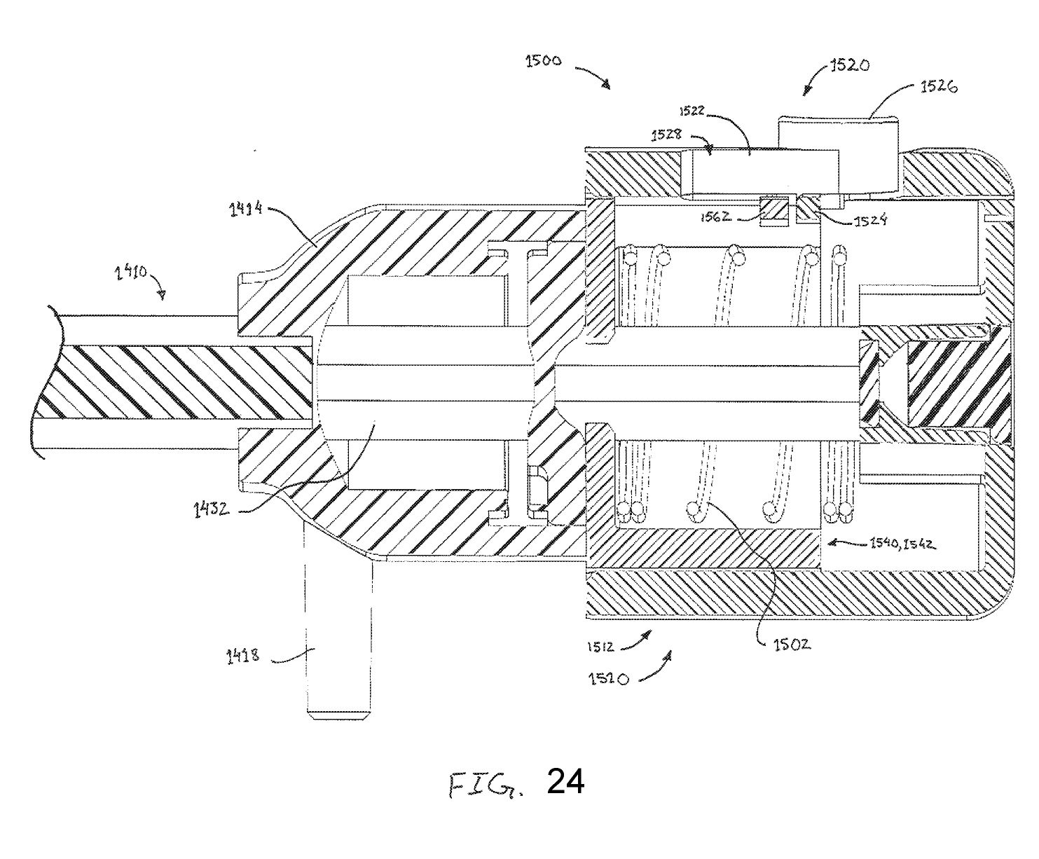

FIG. 24 shows the various components of obturator actuation assembly (1500) when in the initial position. As can be seen, external member (1510) is initially disposed in a proximal position relative to internal member (1510). With external member (1510) disposed proximally relative to internal member (1540), each lock feature (1524) of external member (1510) is engaged with lock members (1562) in lock portion (1566) of internal member (1540). Thus, when external member (1510) is in the proximal position external member (1510) is selectively locked in position to prevent distal movement of obturator (1430) relative to cannula (1410).

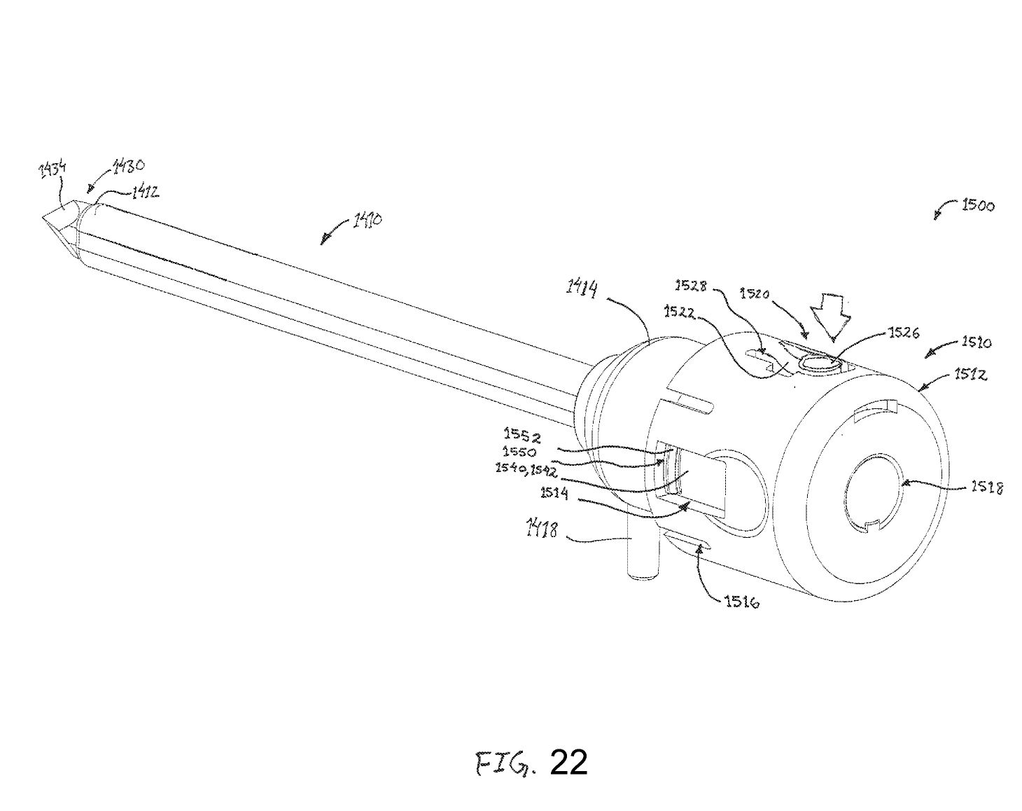

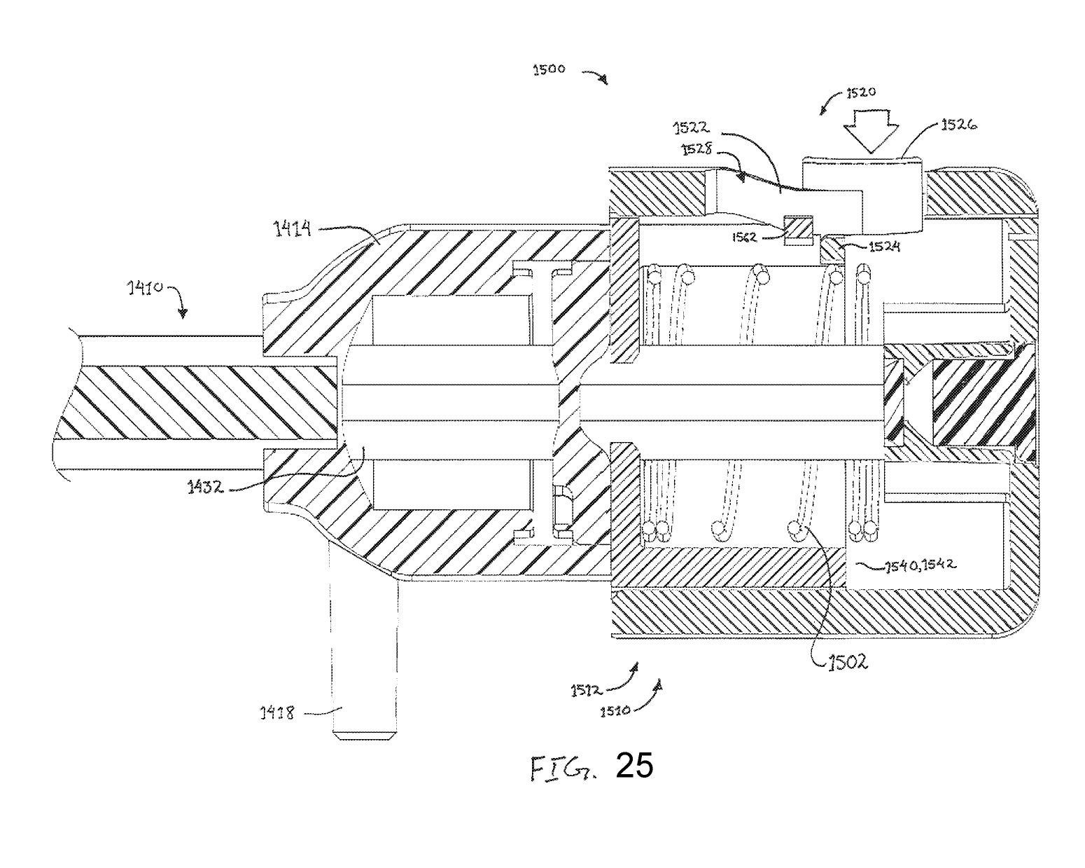

Once an operator has positioned targeting set to the desired depth within tissue of a patient, the operator may desire to further advance obturator (1430) into the tissue of the patient. To advance obturator (1430) relative to cannula (1410) using obturator actuation assembly (1500), an operator may initiate actuation by first unlocking relative movement between obturator (1430) and cannula (1410). To unlock such movement, an operator may apply a force to button (1526) of actuation lock (1520) as shown in FIG. 22. Such a force directs actuation lock (1520) of external member (1510) deforming resilient feature (1522) to deflect lock feature (1524) out of engagement with lock members (1562) of internal member (1540). As can be seen in FIG. 25, such a deflection of resilient feature (1522) causes lock feature (1524) to be positioned below lock members (1562) of internal member (1540). With lock feature (1524) of actuation lock (1520) disengaged from lock members (1562) of internal member (1540), external member (1510) is in an unlocked state and is free to move distally relative to internal member (1540).

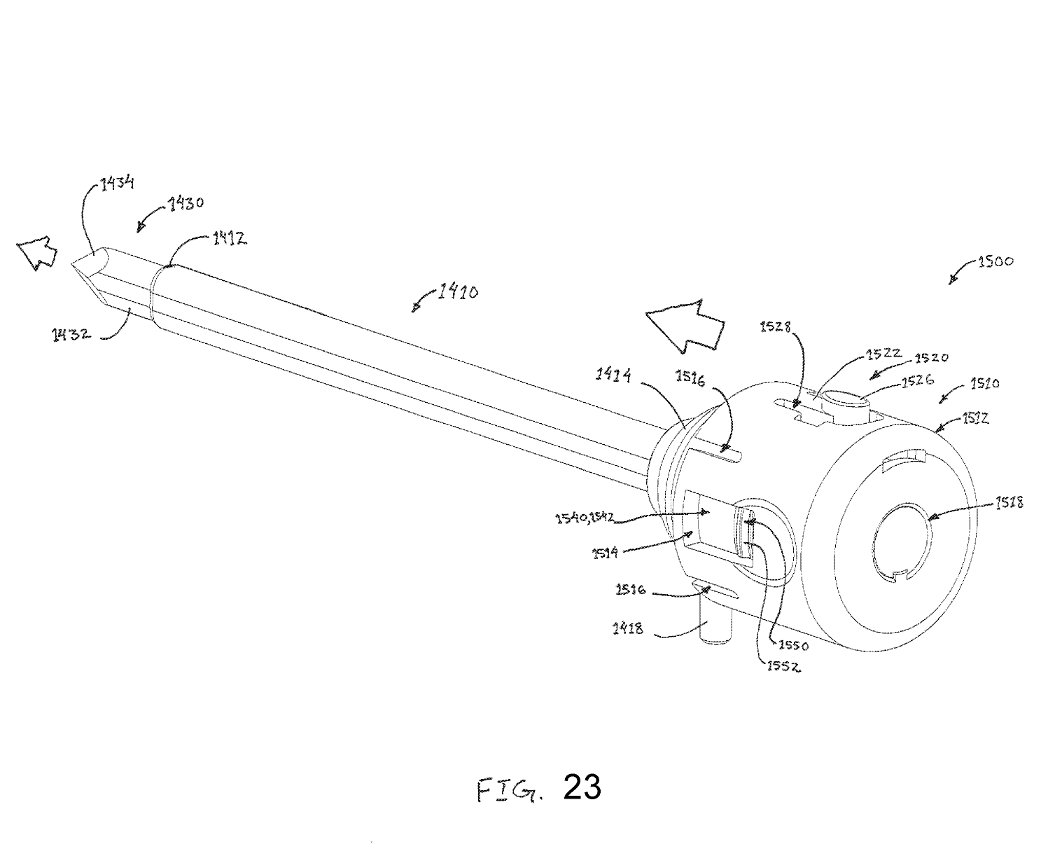

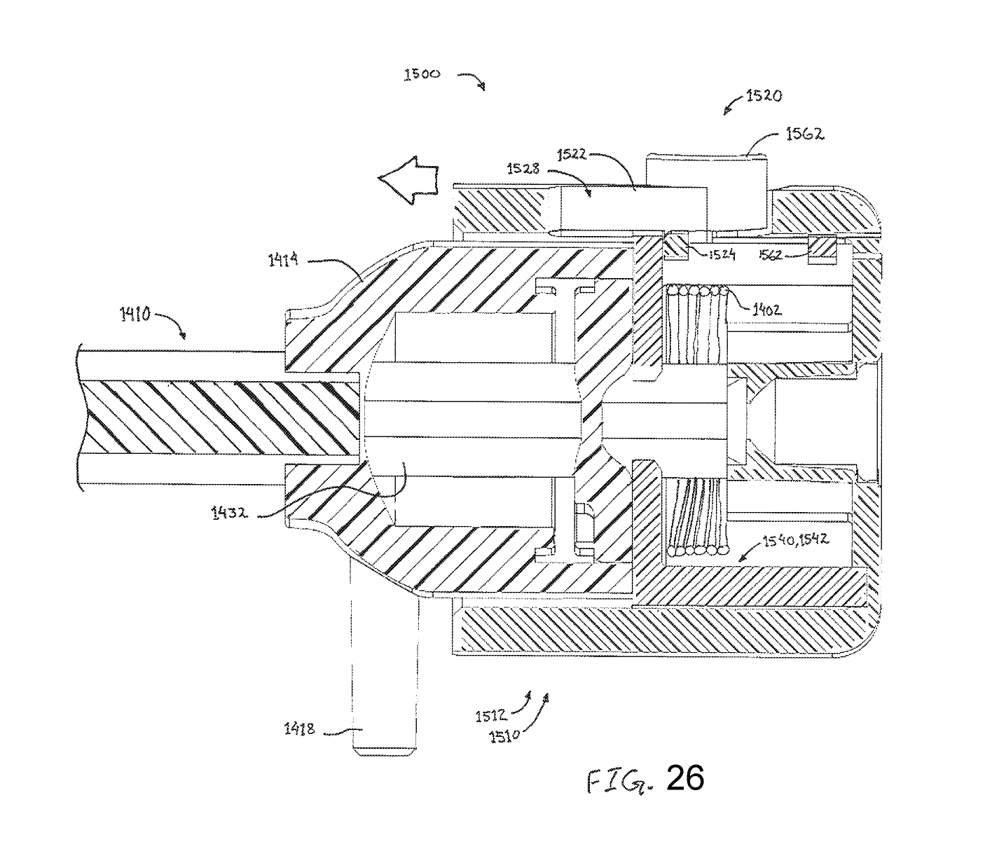

Once external member (1510) is in an unlocked state, an operator may force external member (1510) distally by gripping external member (1510) and applying a distal force to external member (1510) as shown in FIGS. 23 and 26. Such a force will cause external member (1510) to advance obturator (1430) distally relative to cannula (1410). It should be understood that a suitable distal force will be sufficient to overcome any force supplied by spring (1502), which is disposed between internal member (1540) and external member (1510). An operator may advance external member (1510) any suitable distance to a maximum predetermined distance defined by the length of retaining openings (1514) disposed in external member (1510). Once a desired distance of actuation has been reached (or the maximum distance), an operator may optionally hold external member (1510) in position.

Once an operator desires to return actuation assembly (1500) to the initial position, the operator may release external member (1510). Spring (1502), disposed between internal member (1540) and external member (1510), will then force external member (1510) proximally such that lock feature (1524) is positioned distally adjacent to lock members (1562) of internal member (1540). An operator may then return external member (1510) to the position shown in FIGS. 14 and 24 by pressing button (1526) to permit lock feature (1524) to move proximally of lock members (1562). It should be understood that although not shown, in some examples lock feature (1524) and lock members (1562) may include ramps or other features similar to ramped surfaces (1129, 1156) to automatically return external member (1510) to the position shown in FIGS. 14 and 24 without operator intervention. It should also be understood that while the present example uses spring (1502) to return external member (1510) to the proximal position, no such limitation is intended. For instance, in some examples spring (1502) may be omitted and external member (1510) may be manually actuated in both directions.

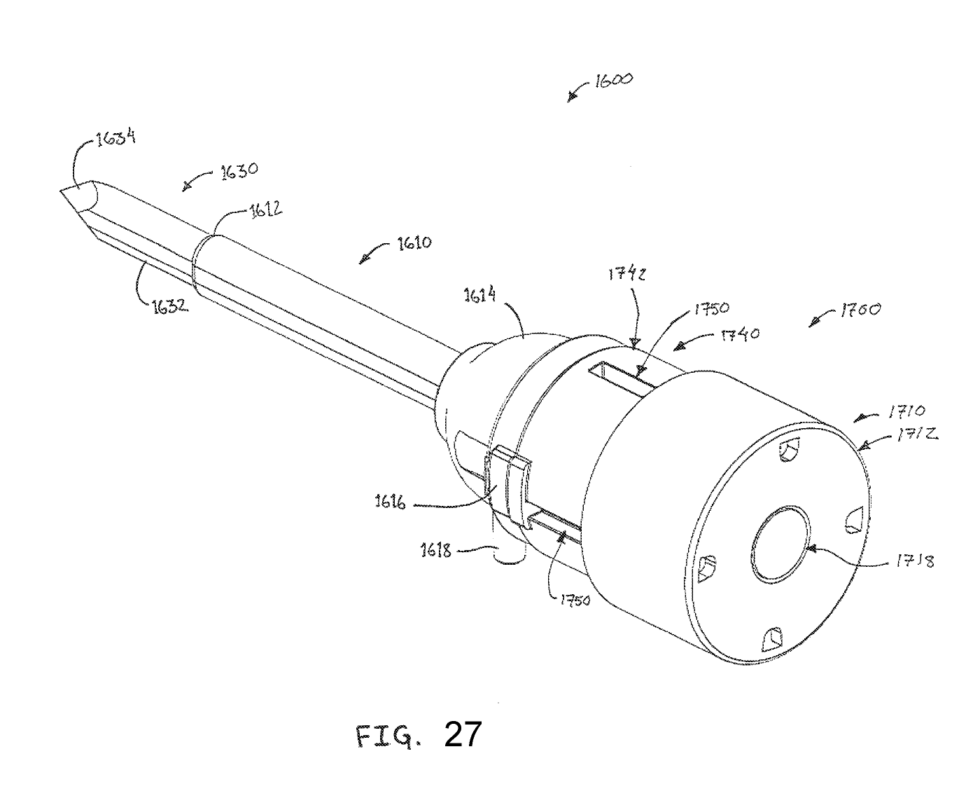

FIGS. 27-33 show still another exemplary alternative targeting set (1600) for use in association with probe (91) as similarly described above with respect to targeting set. Like with targeting set, targeting set (1600) of the present example comprises a cannula (1610), an obturator (1630), and an obturator actuation assembly (1700). As will be described in greater detail below, targeting set (1600) is generally configured such that obturator (1630) is independently actuatable relative to cannula (1610) while cannula (1610) remains longitudinally fixed via a depth stop member (not shown).

Cannula (1610) of the present example defines a lumen (not shown) and includes an open distal end (1612). The proximal end of cannula (1610) is fixedly secured to a hub (1614). Hub (1614) includes attachment features (1616) and a port (1618). As will be described in greater detail below, attachment features (1616) couple hub (1614) to a portion of obturator actuation assembly (1700). Port (1618) is in communication with the lumen defined by cannula (1610). Port (1618) may be optionally coupled to a fluid source for delivery of therapeutic substances, saline, or other fluids to a biopsy site via the lumen. It should be understood that port (1618) is merely optional and may be omitted in some examples. While not shown, it should be understood that hub (1614) includes other features and/or components such as seals, thumbwheels, fluid channels, and/or additional lumen similar to the cylindrical hub described in U.S. Pat. No. 7,507,210.

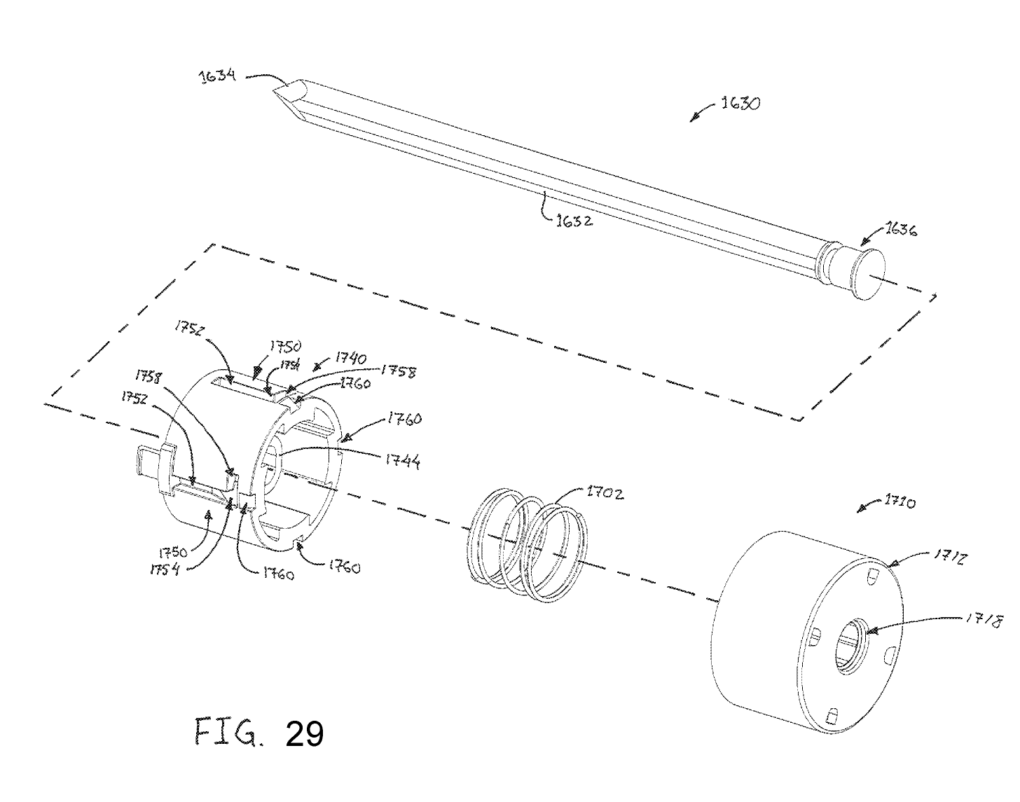

Obturator (1630) of the present example comprises a solid elongate shaft (1632) having a sharp distal tip (1634) and an ovular transverse cross-section. Shaft (1632) of the present example comprises a single MRI compatible material such as ceramic or plastic, although no such limitation is intended. For instance, in other examples shaft (1632) comprises a non-MRI compatible material such as metal. However, in such examples, obturator (1630) may be removed from cannula (1610) during an MRI imaging procedure. As can best be seen in FIG. 29, the proximal end of obturator (1630) includes an attachment feature (1636). As will be described in greater detail below, attachment feature (1636) is configured to be received by at least a portion of obturator actuation assembly (1700) such that obturator (1630) is fixedly secured relative to at least a portion of obturator actuation assembly (1700).

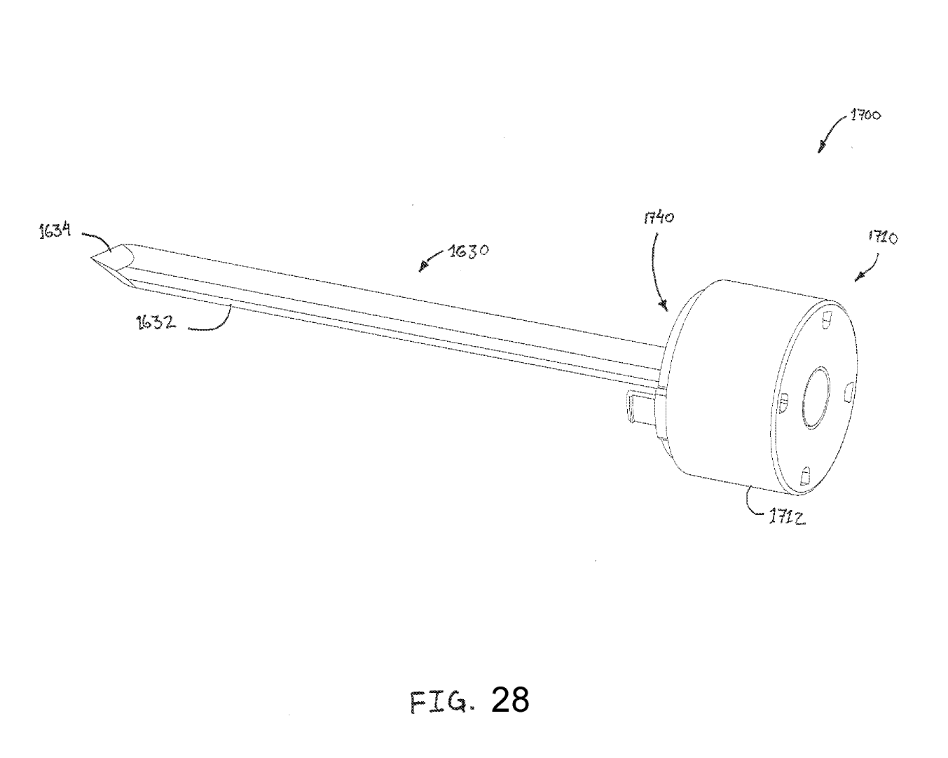

FIGS. 28-33 show obturator (1630) and obturator actuation assembly (1700). As can be seen in FIG. 29, obturator actuation assembly (1700) generally comprises an external member (1710), a spring (1702), and an internal member (1740). As will be described in greater detail below, external member (1710) is generally selectively movable relative to internal member (1740) to selectively actuate obturator (1630) relative to cannula (1610).

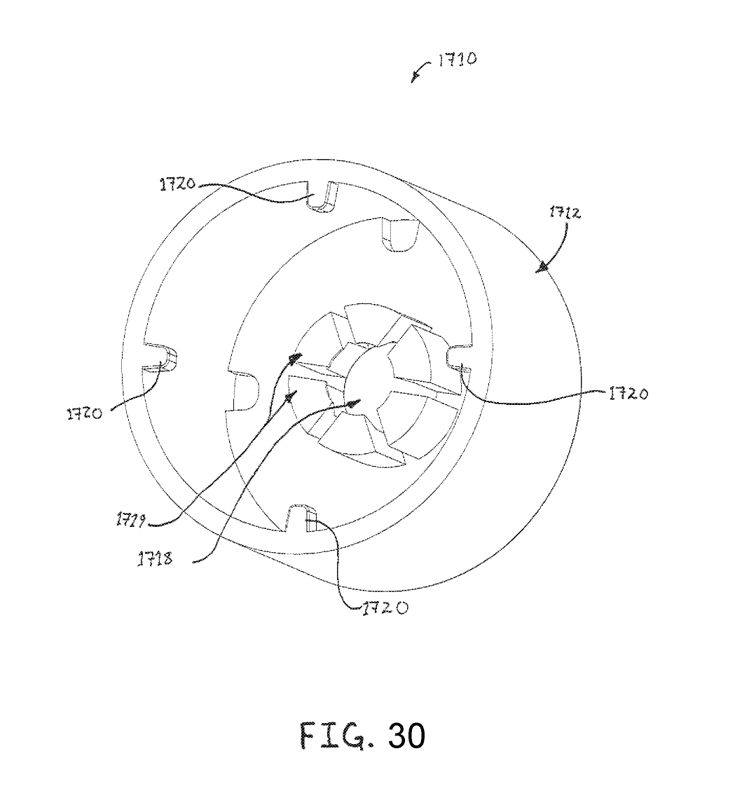

External member (1710) is shown in FIG. 30. Unlike external member (1110) described above with respect to targeting set (1000), external member (1710) is generally configured to move relative to internal member (1740) to actuate obturator (1630). External member (1710) comprises a generally cylindrical hollow body (1712). Body (1712) is generally configured to receive internal member (1740) such that external member (1710) is selectively movable relative to internal member (1740) as will be described in greater detail below.

The proximal end of body (1712) is substantially closed and includes a central bore (1718) disposed at the center point of body (1712). Central bore (1718) is configured to fixedly secure obturator (1630) to external member (1710). In particular, central bore (1718) includes a plurality of attachment features (1719). Attachment features (1719) are configured to engage with corresponding attachment feature (1636) of obturator (1630) to secure obturator (1630) to external member (1710). It should be understood that attachment features (1719) of central bore (1718) are only configured to secure obturator (1630) longitudinally. Accordingly, obturator (1630) remains free to rotate relative to external member (1710). As will be described in greater detail below, such a feature permits external member (1710) to rotate relative to obturator (1639) as actuation assembly (1700) is used to actuate obturator (1630).

The distal end of body (1712) is open and is configured to receive internal member (1740). A plurality of actuation tabs (1720) are positioned at the distal end of body (1712). Actuation tabs (1720) are integral with body and extend inwardly from the inner diameter of body (1712). Each actuation tab (1720) is spaced equidistantly around the inner diameter of body (1712). As will be described in greater detail below, actuation tabs (1720) are generally configured to engage a corresponding portion of internal member (1740) to selectively engage and disengage actuation of actuation assembly (1710). It should be understood that because actuation tabs (1720) of the present example are integral with body (1712), the particular mechanical properties of actuation tabs (1720) are influenced by the particular material of body (1712). For instance, body (1712) of the present example comprises a generally rigid yet elastically deformable plastic. Because body (1712) comprises such a material, it should be understood that actuation tabs (1720) of the present example are similarly rigid yet elastically deformable. Although actuation tabs (1720) are described herein as being integral with body (1712) it should be understood that in other examples actuation tabs (1720) are separate parts fixedly secured to body (1712). Accordingly, in such examples, the particular mechanical properties of actuation tabs (1720) may be varied as desired by merely changing the material of each actuation tab (1720) rather than the material of body (1712).

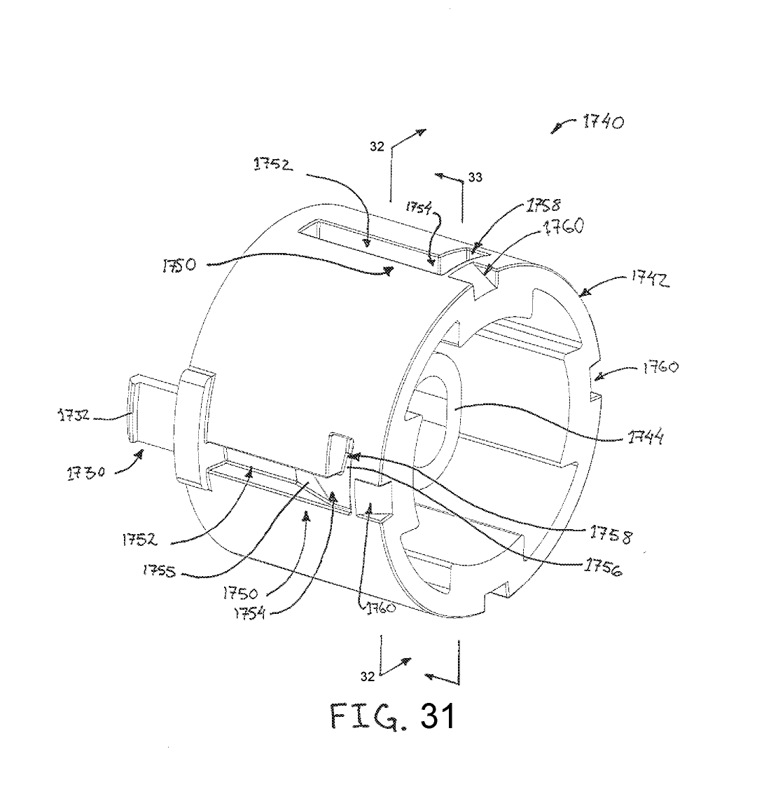

As can best be seen in FIGS. 31-33, internal member (1740) includes a generally rigid hollow cylindrical body (1742) and a pair of attachment arms (1730) extending distally from body (1742). Body (1742) comprises an obturator receiving member (1744), and four actuation slots (1750), Obturator receiving member (1744) is configured to slidably receive obturator (1630). For instance, internal member (1740) of the present example is attachable to hub (1614) of cannula (1610) described above such that cannula (1610) and internal member (1740) remain fixed while obturator (1630) and external member (1710) are advanced relative to cannula (1610) and internal member (1740). Accordingly, obturator receiving member (1744) permits obturator (1630) to slidably extend through internal member (1740) for attachment to external member (1710) as described above.

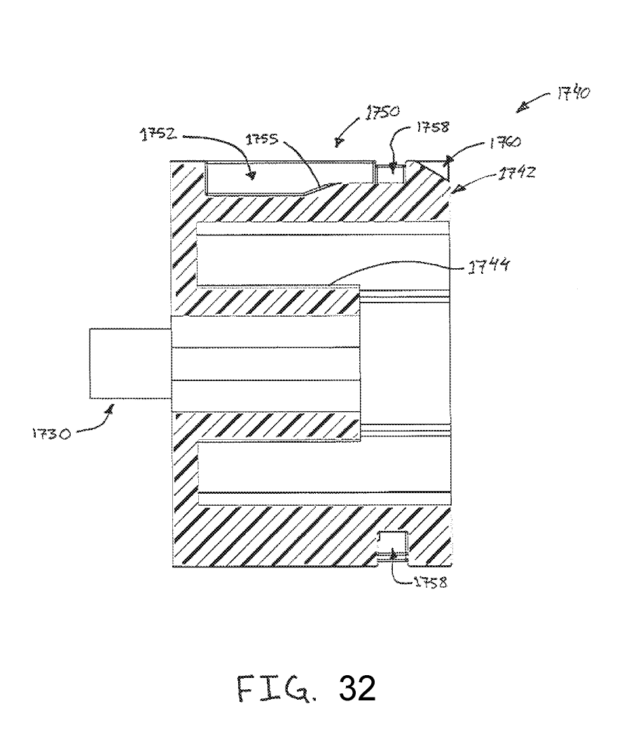

Actuation slots (1750) are generally configured to receive actuation tabs (1720) of external member (1710) to selectively engage and disengage actuation of obturator (1630) via external member (1710). Each actuation slot (1750) includes an actuation portion (1752), a ramp portion (1754), and a lock portion (1758). As can best be seen in FIG. 32, actuation portion (1752) has a generally flat bottom extending distally from ramp portion (1754). Actuation portion (1752) of each actuation slot (1750) is generally configured to slidably receive a respective actuation tab (1720) of external member (1710) to permit external member (1710) to actuate obturator (1630) relative to cannula (1610).

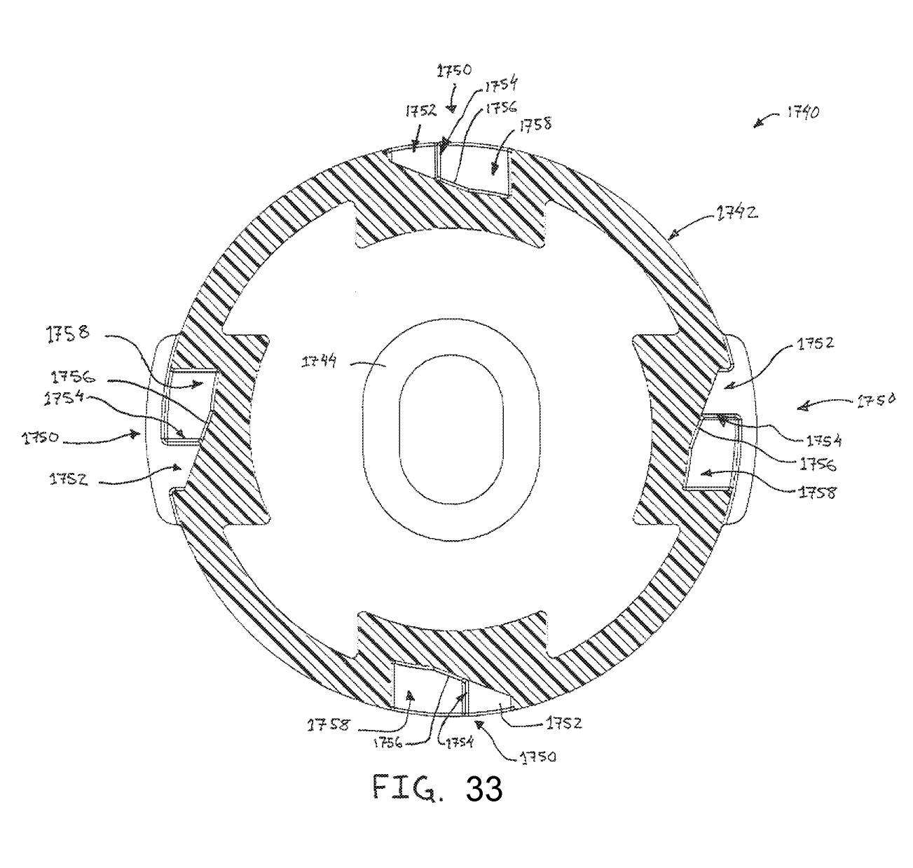

As can best be seen in FIG. 33, lock portion (1758) of each actuation slot (1750) has a generally flat bottom (matching the curvature of internal member (1740)) extending radially to ramp portion (1754). As will be described in greater detail below, lock portion (1758) of each actuation slot (1750) is generally configured to receive a respective actuation tab (1720) of external member (1710) to selectively prevent actuation of actuation assembly (1700).

Ramp portion (1754) of each actuation slot (1750) is disposed between actuation portion (1752) and lock portion (1758) with actuation portion (1752) and lock portion (1758) extending away from ramp portion (1754) orthogonally relative to each other. Ramp portion (1754) comprises an actuation ramp (1755) and a lock ramp (1756). Actuation ramp (1755) is adjacent to actuation portion (1752) and is configured to direct a respective actuation tab (1720) into actuation portion (1752) when actuation tab (1720) is disposed on actuation ramp (1755). Similarly, lock ramp (1756) is adjacent to lock portion (1758) and is configured to direct a respective actuation tab (1720) into lock portion (1758) when actuation tab (1720) is disposed on actuation ramp (1755). Thus, it should be understood that each actuation tab (1720) is directable by ramp portion (1754) to either actuation portion (1752) or lock portion (1758). It should be also understood that each actuation tab (1720) is generally resilient such that each actuation tab (1720) resiliently engages ramp portion (1754) to further direct actuation tab (1720) to either actuation portion (1752) or lock portion (1758).

The proximal end of internal member (1740) further includes a plurality of chamfered portions (1760) oriented adjacently to each actuation slot (1750). Chamfered portions (1760) are generally configured for assembly purposes. For instance, chamfered portions (1760) are configured to receive each actuation tab (1720) of external member (1710) as internal member (1740) is inserted into external member (1710), thereby compressing each actuation tab (1720) inwardly over the exterior of internal member (1740).

As described above, attachment arms (1730) extend distally from body (1742) of internal member (1740). Attachment arms (1730) are configured to engage with attachment features (1616) disposed on hub (1614) of cannula (1610). In particular, each arm (1730) includes outwardly extending teeth (1732) that are resiliently biased to fasten arms (1730) into attachment features (1616). Thus it should be understood that arms (1730) secure internal member (1740) to cannula (1610) such that movement of external member (1710) and cannula (1610) is fixed relative to each other.

FIGS. 27 and 34-39 show an exemplary use of obturator actuation assembly (1700) to actuate obturator (1630) relative to cannula (1610). In particular, as can be seen in FIG. 27, obturator actuation assembly (1700) may begin in an initial state where obturator (1630) is in a proximal position relative to cannula (1610). In the initial state an operator may use targeting set (1600) in conjunction with depth stop device (95) and guide cube (104) to penetrate into tissue of a patient to a desired initial depth, as similarly described above with respect to targeting set.

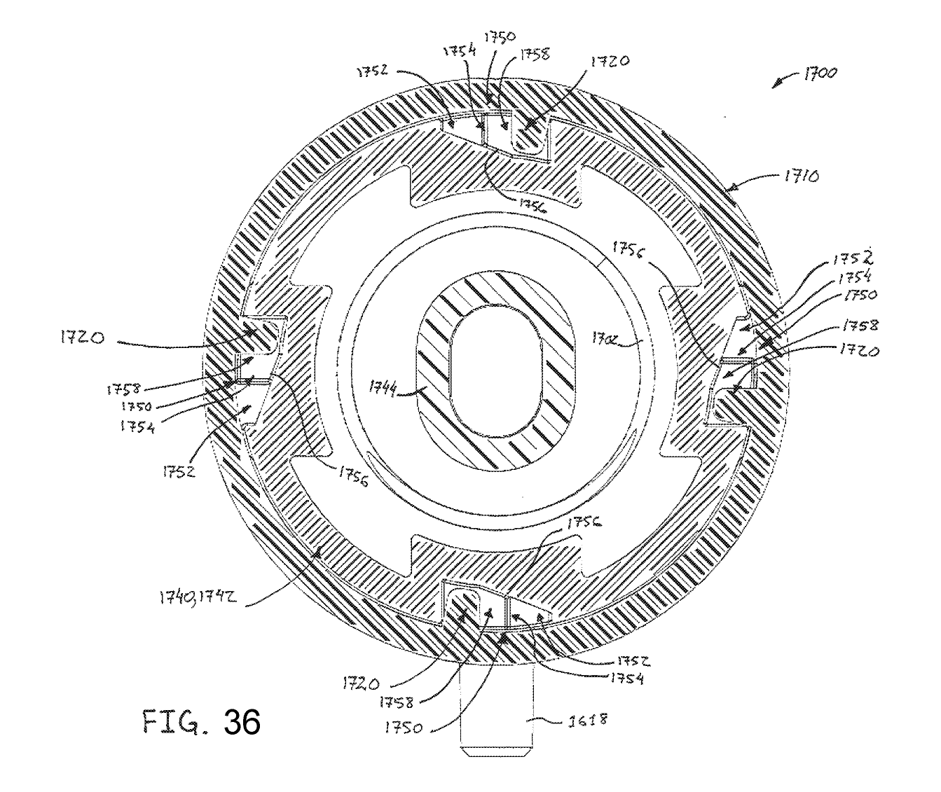

FIG. 36 shows the various components of obturator actuation assembly (1700) when in the initial position. As can be seen, external member (1710) is initially disposed in a proximal position relative to internal member (1740). With external member (1710) disposed proximally relative to internal member (1740), each actuation tab (1720) of external member (1710) is disposed within lock portion (1758) of each respective actuation slot (1750) in internal member (1540). When external member (1710) is in this position, external member (1710) can only rotate in the counter clockwise direction relative to internal member (1740) because lock portion (1758) prevents articulation tabs (1720) from moving distally or rotating in the clockwise direction. Thus, when external member (1710) is in the proximal position external member (1710) is selectively locked in position to prevent distal movement of obturator (1630) relative to cannula (1610).

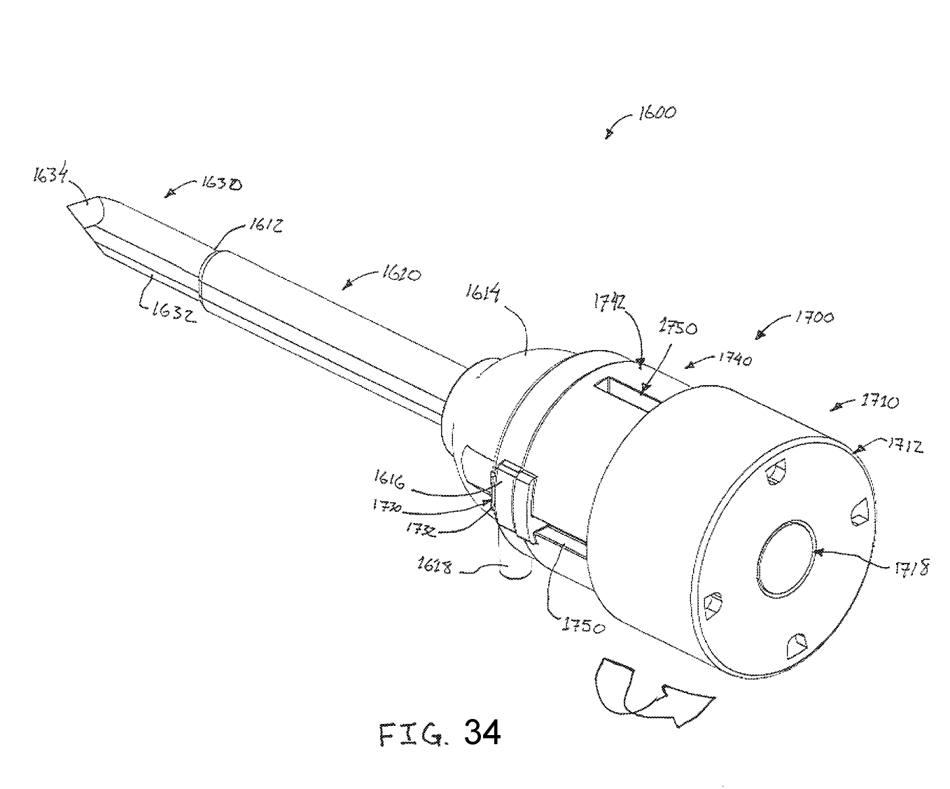

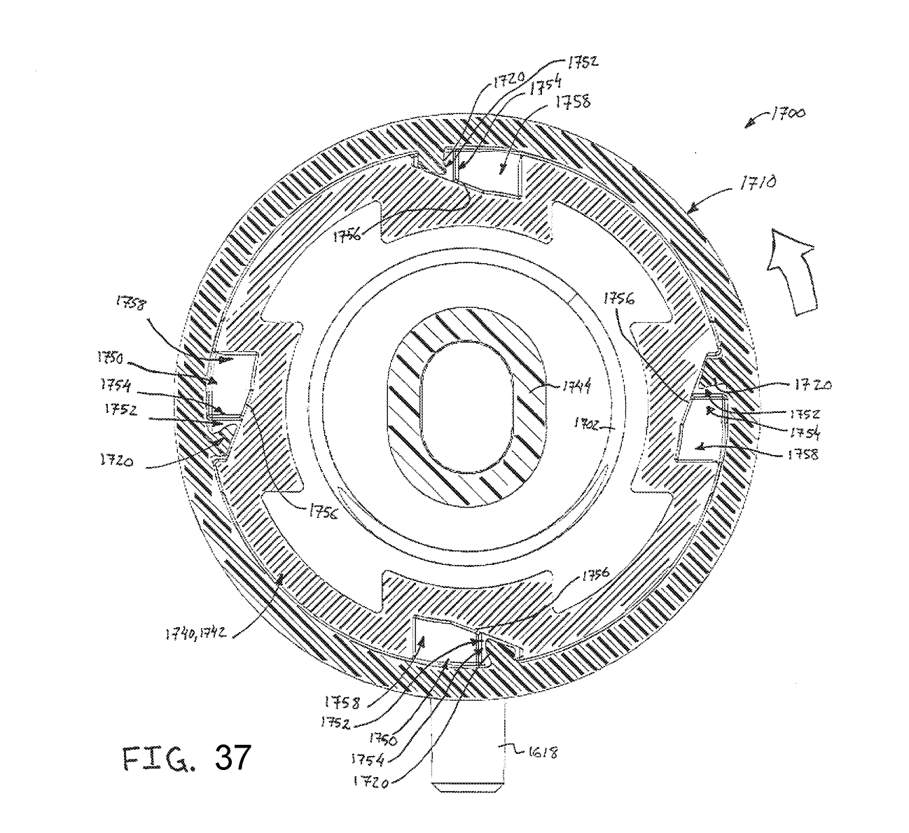

Once an operator has positioned targeting set to the desired depth within tissue of a patient, the operator may desire to further advance obturator (1630) into the tissue of the patient. To advance obturator (1630) relative to cannula (1610) using obturator actuation assembly (1700), an operator may initiate actuation by first unlocking relative movement between obturator (1630) and cannula (1610). To unlock such movement, an operator may apply a force to external member (1710) to rotate external member (1710) in the counter clockwise direction as shown in FIG. 34. Such a force directs actuation tabs (1720) of external member (1710) out of lock portion (1758) of actuation slot (1750) in internal member (1740) and into ramp portion (1754) of actuation slot (1750). As actuation tabs (1720) encounter lock ramp (1756), actuation tabs (1720) resiliently deform until actuation ramp (1755) is reached as seen in FIGS. 36 and 37. Once external member (1710) has been rotated to position actuation tabs (1720) in a position adjacent to actuation ramp (1755) of actuation slot (1750), actuation tabs (1720) resiliently bear against actuation ramp (1755) driving actuation tabs (1720) down actuation ramp (1755) and into actuation portion (1752) of actuation slot (1750).

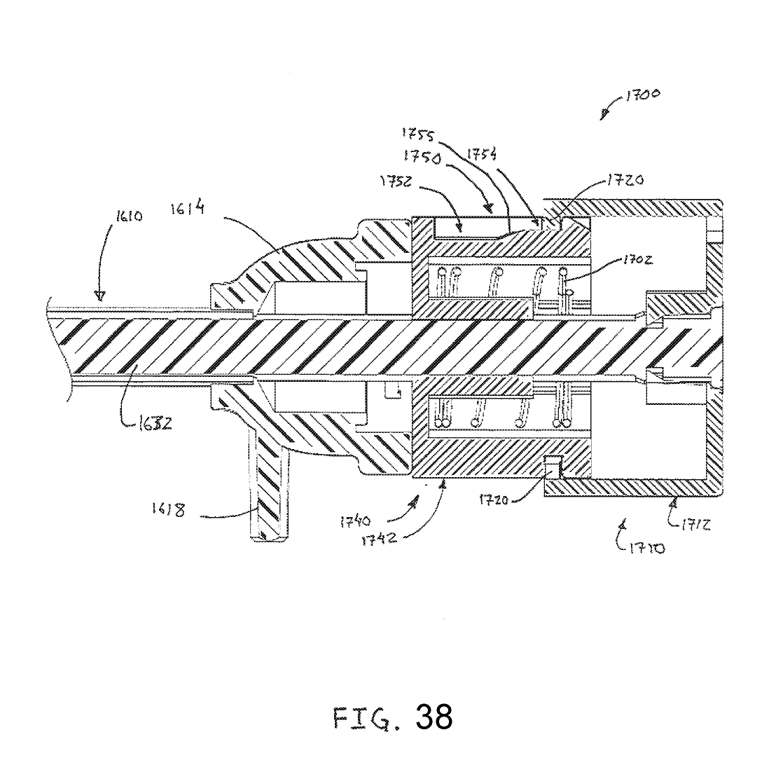

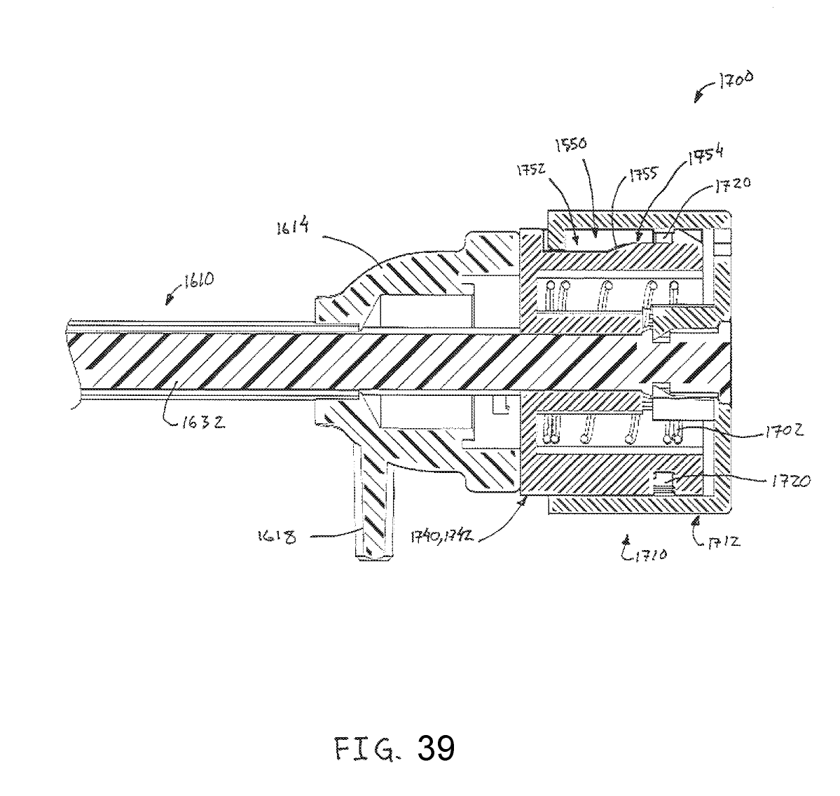

With actuation tabs (1720) positioned in actuation portion (1752) of actuation slot (1750), external member (1710) is free to move distally to actuate obturator (1630) relative to cannula (1610). As shown in FIGS. 38 and 39, an operator may next initiate actuation of obturator (1630) by gasping external member (1710) and driving external member (1710) distally relative to internal member (1740).

Once an operator desires to return actuation assembly (1700) to the initial position, the operator may release external member (1710). Spring (1702), disposed between internal member (1740) and external member (1710), will then force external member (1710) proximally such that actuation tabs (1720) travel proximally through actuation portion (1752) of actuation slot (1750) and up actuation ramp (1755) of ramp portion (1754). Once actuation tabs (1720) travel up actuation ramp (1755), actuation tabs (1720) resiliently bear against lock ramp (1756) driving actuation tabs (1720) down lock ramp (1756), thereby returning external member (1710) to the initial proximal position. It should be understood that while the present example uses spring (1702) to return external member (1710) to the proximal position, no such limitation is intended. For instance, in some examples spring (1702) may be omitted and external member (1710) may be manually actuated in both directions.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometric s, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus for use with a biopsy device to position a needle of the biopsy device within breast tissue, the apparatus comprising:
(a) a cannula comprising an open distal end and a longitudinal lumen, wherein the longitudinal lumen extends through the cannula and is in communication with the open distal end, wherein the longitudinal lumen is sized to receive the needle of the biopsy device;
(b) an obturator sized for insertion into the longitudinal lumen of the cannula, the obturator having a distal end extending from the open distal end of the cannula when the obturator is inserted into the cannula, and the obturator having a recess proximate to the distal end of the obturator; and
(c) an obturator actuation assembly, wherein the obturator actuation assembly is secured to a proximal end of the obturator, wherein the hub assembly is configured to selectively actuate the obturator between a first position and a second position relative to the cannula.

2. The apparatus of claim 1, further comprising a hub, wherein the hub is secured to the cannula, wherein the hub is configured to selectively fasten to at least a portion of the obturator actuation assembly.

3. The apparatus of claim 2, wherein the obturator actuation assembly comprises a first member and a second member, wherein the first member is secured to the obturator, wherein the second member is secured to the hub.

4. The apparatus of claim 3, wherein the first member is selectively movable relative to the second member to actuate the obturator between the first position and the second position.

5. The apparatus of claim 2, wherein the hub comprises a fluid port, wherein the fluid port is configured to communicate fluids to the longitudinal lumen of the cannula.

6. The apparatus of claim 1, wherein the obturator actuation assembly includes a resilient feature, wherein the resilient feature is configured to resiliently bias the obturator toward the first position.

7. The apparatus of claim 6, wherein the resilient feature comprises a coil spring.

8. The apparatus of claim 1, further comprising a lock feature, wherein the lock feature is configured to selectively retain the obturator actuation assembly in the first position.

9. The apparatus of claim 1, wherein the obturator is substantially solid.

10. The apparatus of claim 1, wherein the cannula includes a lateral aperture disposed proximally of the open distal end, wherein the obturator includes a lateral recess.

11. The apparatus of claim 10, wherein the lateral recess of the obturator is configured to align with the lateral aperture of the cannula when the obturator is in the first position.

12. The apparatus of claim 1, wherein the cannula comprises an MRI compatible material.

13. The apparatus of claim 12, wherein the obturator comprises an MRI compatible material.

14. The apparatus of claim 1, wherein the obturator defines an oval shaped cross-section.

15. The apparatus of claim 1, wherein the obturator comprises a sharp distal tip, wherein the sharp distal tip is disposed distally of the open distal end of the cannula when the obturator is disposed within the cannula.

16. An apparatus for use with a biopsy device to position a needle of the biopsy device within breast tissue, the apparatus comprising:
(a) a cannula comprising an open distal end, wherein the cannula further defines a lateral opening, wherein the lateral opening is proximate to the open distal end of the cannula, wherein the cannula further comprises a longitudinal lumen communicating with the lateral opening and the open distal end, wherein the longitudinal lumen is sized to receive the needle of the biopsy device;
(b) an obturator sized for insertion into the longitudinal lumen of the cannula, the obturator having a sharp distal end extending from the open distal end of the cannula when the obturator is inserted into the cannula, and the obturator having a recess proximate to the distal end of the obturator; and
(c) a hub assembly, wherein the hub assembly is secured to the distal end of the cannula and the obturator, wherein the hub assembly is configured to selectively drive the obturator between a first position and a second position relative to the cannula.

17. The apparatus of claim 16, wherein the obturator defines a first distance between the sharp distal end of the obturator and the open distal end of the cannula when the obturator is in the first position.

18. The apparatus of claim 17, wherein the obturator further defines a second distance between the sharp distal end of the obturator and the open distal end of the cannula when the obturator is in the second position.

19. The apparatus of claim 18, wherein the second distance is greater than the first distance.

20. A method for positioning an obturator and cannula into tissue of a patient, the method comprising the steps of:
(a) inserting the obturator into a lumen defined by the cannula such that a sharp distal tip of the obturator protrudes through an open distal end of the cannula, wherein the act of inserting the obturator into the lumen of the cannula defines a first distance between the sharp distal tip of the obturator and the open distal end of the cannula;
(b) piercing tissue of a patient by inserting the obturator into tissue of the patient, wherein the obturator is inserted into tissue of the patient while the obturator is disposed within the lumen of the cannula; and
(c) actuating the obturator relative to the cannula to drive the obturator toward a second position, wherein the obturator in the second position defines a second distance between the sharp distal tip of the obturator and the open distal end of the cannula, wherein the second distance is greater than the first distance.
